(19) United States
(12) Patent Application Publication
(10) Pub. No.: US 2024/0002800 A1
(43) Pub. Date: Jan. 4, 2024

(54) USE OF LEUKEMIA-DERIVED CELLS FOR ENHANCING NATURAL KILLER (NK) CELL THERAPY

(71) Applicant: Mendus B.V., Leiden (NL)

(72) Inventors: Alex Karlsson-Parra, Leiden (NL); Erik Hans Manting, Leiden (NL); Haoxiao Zuo, Leiden (NL); Satwinder Kaur Singh, Leiden (NL)

(21) Appl. No.: 18/197,670

(22) Filed: May 15, 2023

Related U.S. Application Data
(60) Provisional application No. 63/382,598, filed on Nov. 7, 2022.
(60) Provisional application No. 63/342,396, filed on May 16, 2022.

(51) Int. Cl. C12N 5/0783

(52) U.S. Cl. CPC C12N 5/0646; C12N 2501/2302; C12N 2501/2321; C12N 2501/2315

(57) ABSTRACT

The present disclosure provides ex vivo methods which employ modified cells of leukemic origin to enhance the efficacy of adoptive cell therapy with natural killer (NK) cells.

### RELATED APPLICATIONS

This application claims priority to U.S. Provisional Patent Application Serial Nos. 63/342,396, filed May 16, 2022, and 63/382,598, filed Nov. 7, 2022, the entire disclosures of which are hereby incorporated herein by reference.

### BACKGROUND

Natural killer (NK) cells are an important component of the human immune system. In humans, the stable expansion of a defined subset within circulating NK cells has been solidly documented in a large fraction of healthy, human cytomegalovirus (CMV)-seropositive individuals (Rolle et al, 2016). This subset, named “memory” (or “adaptive”) NK cells, is distinct from conventional NK cells in regard to the expression of certain surface markers. Among their phenotypic characteristics, memory NK cells preferentially express NKG2C, the HLA-E-specific activating receptor, associated with the CD57 terminal maturation marker (Rolle et al, 2016). Memory NK cells' most relevant functional feature is represented by a markedly higher functional response to CD16 stimulation, including ADCC and IFN-gamma/TNF-alpha production (Schlums et al, 2016; Lee et al, 2015; Hwang et al, 2012).

Several features render memory NK cells a potentially attractive contributor to the efficacy of mAb-based therapeutic strategies (Capuano et al, 2019). The capability of memory NK cells to provide an amplified functional response to CD16 cross-linking upon interaction with antibody-coated target cells is particularly relevant in this regard (Schlums et al, 2015; Lee et al, 2015; Kim et al, 2013) and may impact the clinical efficacy of tumor-targeting mAb therapies. However, due to their more differentiated phenotype, memory NK cells proliferate poorly when standard expansion protocols are used. Accordingly, new methods are needed to enhance the efficacy of NK cell therapy.

### SUMMARY

The present disclosure provides findings that modified cells of leukemic origin can improve the viability, expansion, efficacy and/or functionality of certain immune cells (e.g., autologous patient derived NK cells or NK cells from a donor) employed in adoptive cell therapy when these cells are combined together ex vivo. In certain embodiments, natural killer (NK) cells (e.g., memory NK cells) that are co-cultured in the presence of the cells of leukemic origin exhibit improved expansion and persistence following subsequent administration to a patient by adoptive cell transfer. In certain embodiments, the NK cells may exhibit prolonged post-infusion survival due to prior co-culturing ex vivo with the modified cells of leukemic origin. Accordingly, the methods of the present disclosure address one of the main bottlenecks in adoptive NK cell therapies, namely the limited expansion capacity of NK cells, particularly for patient derived autologous NK cells.

In certain aspects, a method is disclosed for activating, stimulating and and/or expanding natural killer (NK) cells, comprising: (a) obtaining a population of immune cells comprising natural killer (NK) cells; (b) contacting the population of immune cells with a modified cell of leukemic origin, wherein the modified cell exhibits a mature dendritic cell phenotype; and (c) co-culturing the population of immune cells with the modified cell of leukemic origin, wherein the modified cell of leukemic origin activates the natural killer (NK) cells in the population of immune cells, thereby expanding the natural killer (NK) cells.

In certain exemplary embodiments, the NK cells express NKG2C on their cell surface. In certain exemplary embodiments, the NK cells are memory NK cells expressing NKG2C and CD57 on cell surface. In certain exemplary embodiments, the NK cells are precursor memory (NKG2C+/CD57−) NK cells expressing NKG2C but not CD57 on cell surface. In certain exemplary embodiments, the NK cells are low FcεRIγ expression memory NK cells. In certain exemplary embodiments, the expanded population of activated NK cells predominantly comprises FcεRIγ negative memory NK cells, i.e., the percentage or frequency of FcεRIγ expression is low in the total NK cells. In certain embodiments, the NK cells are FcεRIγ negative memory NK cells.

In certain aspects, the population of NK cells are autologous NK cells from a patient (subject) suffering from a cancer. In other aspects, the NK cells are allogeneic NK cells from one or more healthy donors.

In certain exemplary embodiments, the population of immune cells comprise engineered NK cells. For instance, the NK cells may be modified to express an engineered immune receptor selected from a chimeric antigen receptor (CAR) or a T cell receptor (TCR) which binds a tumor antigen in the patient. See e.g., Mensali et al., NK cells specifically TCR-dressed to kill cancer cells. Lancet, Vol. 40, P106-117 (2019).

In other aspects, the modified cell of leukemic origin exhibits a mature dendritic cell phenotype and is non-proliferating.

In other aspects, the population of immune cells (e.g., NK cells) and the modified cell of leukemic origin are co-cultured under conditions suitable to stimulate proliferation and activation of the immune cells, thereby generating the population of immune cells with increased viability and enhanced activation status.

In certain exemplary embodiments, the disclosure provides methods for treating a patient suffering from the cancer, the method further comprising administering the population of immune cells (e.g., NK cells with enhanced activation status) to the patient suffering from the cancer. In certain embodiment, the NK cells are autologous NK cells obtained from the patient. In other embodiments, the NK cells are allogeneic cells obtained from one or more donor subjects.

In certain exemplary embodiments, the population of immune cells is primarily comprised of NK cells. For example, at least 50% of the population of immune cells (e.g., at least 60%, at least 70%, at least 80%, at least 90%, at least 95%, at least 98% or more of the populations of immune cells) is comprised of NK cells (e.g., memory NK cells). In certain embodiments, other immune cells besides NK cells (e.g., T cells) may be present. For example, T cells may constitute between 0.01% and 30% by cell counts of the population of immune cells. In certain exemplary embodiments, the presence of these T cells facilitates activation of the NK cells by the modified cell of leukemic origin.

In certain exemplary embodiments, the population of the immune cells is co-cultured in the presence of at least one growth factor, optionally, wherein the at least one growth factor is selected from serum, insulin, IFNγ, interleukin-2 (IL-2), IL-4, IL-7, IL-12, IL-15, IL-18, IL-21, GM-CSF, TNF-α, or any combination thereof.

In certain exemplary embodiments, the modified cells of leukemic origin and the NK cells are co-cultured in the presence of certain cytokines. For instance, in certain exemplary embodiments, the modified cells of leukemic origin and the NK cells are co-cultured in the presence of IL-2, IL-15, IL-21, or a combination thereof. In certain exemplary embodiments, the concentration of IL-2 in the co-culture is about 10 IU/ml-6000 IU/mL, about 50 IU/ml-200 IU/mL, or about 100 IU/mL. In certain exemplary embodiments, the concentration of IL-15 in the co-culture is about 5 ng/mL-150 ng/mL, about 50 ng/ml-150 ng/milord about 150 ng/mL. In certain exemplary embodiments, the concentration of IL-21 in the co-culture is about 5 ng/mL-150 ng/mL, about 50 ng/ml-150 ng/milord about 150 ng/mL.

In certain exemplary embodiments, the expanded population of activated NK cells is used for administering to a subject in need thereof.

In certain exemplary embodiments, the methods of the disclosure, further comprise the step of administering activated NK cells to a subject in need thereof.

In certain exemplary embodiments, the population of immune cells such as NK cells are isolated from peripheral blood mononuclear cells (PBMCs) of a subject to whom the expanded population of activated NK cells who is administered. In certain exemplary embodiments, the NK cells are isolated from iPSCs, cord blood or NK cell lines.

In certain exemplary embodiments, the population of immune cells such as NK cells are isolated from peripheral blood mononuclear cells (PBMCs) of an allogeneic third-party donor. In certain exemplary embodiments, the third-party donor is positive for Cytomegalovirus (CMV+).

In certain exemplary embodiments, the modified cell of leukemic origin comprises at least one tumor antigen selected from the group consisting of WT-1, RHAMM, PRAME, MUC-1, p53, and Survivin.

In certain exemplary embodiments, the modified cell of leukemic origin is CD34-positive, CD1a-positive, CD83-positive, and CD14-negative.

In certain exemplary embodiments, the modified cell of leukemic origin comprises a co-stimulatory molecule. In certain embodiments, the co-stimulatory molecule can be selected from an MHC class I molecule, B and T lymphocyte attenuator (BTLA), and a Toll ligand receptor. In certain embodiments, the co-stimulatory molecule can be selected from the co-stimulatory molecule is selected from CD112, CD155, CD70, CD80, CD86, 4-1BBL (CD137-ligand), OX40L, CD30L, CD40, PD-L1, ICOSL, ICAM-1, lymphocyte function-associated antigen 3 (LFA3 (CD58)), K12/SECTM1, LIGHT, HLA-E, B7-H3, and CD83. In certain embodiments, the co-stimulatory molecule is selected from CD112, CD155, and/or CD58.

In certain exemplary embodiments, the modified cell of leukemic origin further comprises a cell surface marker selected from the group consisting of DC-SIGN, Langerin, CD40, CD70, CD80, CD86, and any combination thereof.

In certain exemplary embodiments, the modified cell of leukemic origin is CD70-positive, CD80-positive, and CD86-positive.

In certain exemplary embodiments, the modified cell of leukemic origin comprises an MHC class I molecule.

In certain exemplary embodiments, the modified cell of leukemic origin comprises an MHC class II molecule.

In certain exemplary embodiments, the modified cell of leukemic origin is non-proliferating.

In certain exemplary embodiments, the method further includes a step (c) of administering the expanded population of activated NK cells to a subject.

In certain exemplary embodiments, the subject has been administered an anti-tumor IgG1 antibodies prior to step (c) above or at the same time as step (c).

In certain exemplary embodiments, the NK cells increase antibody-dependent cellular cytotoxicity (ADCC) after administration into the cancer patient.

In certain exemplary embodiments, the NK cells that are co-cultured with modified cells of leukemic origin survive longer after being administered to the subject as compared to NK cells that have not been in contact with the modified cell of leukemic origin prior to administration.

In certain exemplary embodiments, the method further includes a step of introducing a chimeric antigen receptor (CAR) or an engineered T cell receptor (TCR) into the expanded NK cell population to generate a population of engineered NK cells. In certain embodiments, the CAR or the engineered TCR is introduced to the NK cells prior to, during, or subsequent to co-culturing the population of immune cells with the modified cells of leukemic origin.

In certain exemplary embodiments, the CAR or the engineered TCR is specific for one or more tumor antigens present in the subject to receive the expanded population of activated NK cells.

In certain exemplary embodiments, the population of immune cells such as NK cells is capable of reacting with tumor cells of the patient that do not express the tumor antigen to which the engineered immune receptor binds.

In certain exemplary embodiments, the modified cell comprises at least one tumor antigen selected from the group consisting of WT-1, RHAMM, PRAME, MUC-1, p53, and Survivin.

In certain exemplary embodiments, the immune cells are activated following exposure to the endogenous cells expressed by the modified cell of leukemic origin.

In certain exemplary embodiments, the modified cell comprises a genetic aberration between chromosome 11p15.5 to 11p12. In certain exemplary embodiments, the genetic aberration encompasses about 16 Mb of genomic regions.

In certain exemplary embodiments, the modified cell has been irradiated.

In certain exemplary embodiments, the NK cell is an NK CAR cell comprising a chimeric antigen receptor (CAR). In particular embodiments, the CAR comprises an antigen binding domain, a transmembrane domain, and an intracellular domain comprising a costimulatory domain and a primary signaling domain. In certain exemplary embodiments, the antigen binding domain comprises a full-length antibody or antigen-binding fragment thereof, a Fab, a single-chain variable fragment (scFv), or a single-domain antibody. In certain exemplary embodiments, the antigen binding domain is specific for a tumor-associated antigen (TAA) or a non-tumor-associated antigen.

In certain exemplary embodiments, the CAR of the NK CAR further comprises a hinge region. In certain exemplary embodiments, the hinge region is a hinge domain selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof. In certain exemplary embodiments, the transmembrane domain is selected from the group consisting of an artificial hydrophobic sequence, a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS (CD278), or CD154, and a transmembrane domain derived from a killer immunoglobulin-like receptor (KIR). In certain exemplary embodiments, the intracellular domain comprises a costimulatory signaling domain and an intracellular signaling domain. In certain exemplary embodiments, the costimulatory signaling domain comprises one or more of a costimulatory domain of a protein selected from the group consisting of proteins in the TNFR superfamily, CD27, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof. In certain exemplary embodiments, the intracellular signaling domain comprises an intracellular domain selected from the group consisting of cytoplasmic signaling domains of a human CD3 zeta chain (CD3ζ), FcγRIIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In other aspects, a method for treating a disease or disorder in a subject in need thereof, comprising: administering to the subject a population of immune cell such as NK cells produced by any one of the methods disclosed herein.

In certain exemplary embodiments, the disease or disorder is a cancer. In other exemplary embodiments, the disease or disorder is an infectious disease.

In certain exemplary embodiments, the cancer is a tumor. In certain exemplary embodiments, the tumor is a liquid tumor. In certain exemplary embodiments, the tumor is a solid tumor.

In certain exemplary embodiments, the immune cell is a natural killer (NK) cell. In certain exemplary embodiments, the immune cell is an autologous NK cell.

In certain exemplary embodiments, the activation and proliferation of the memory NK cells is effected and/or maintained in the absence anti-tumor antibody-opsonized tumor cells. For instance, in certain embodiments, no anti-tumor antibodies (e.g., anti-PD-L1 or anti-CD20 antibodies) are employed in the disclosed methods. In certain exemplary embodiments, the activation and proliferation of the memory NK cells is effected in the absence anti-tumor antibody-opsonized tumor cells.

In certain exemplary embodiments, the activation and proliferation of the memory NK cells is effected in the absence of cells expressing a ligand for NKG2C on the cell surface of the NK cells. In certain exemplary embodiments, the ligand for NKG2C is HLA-E.

In certain exemplary embodiments, the expanded population of activated NK cells predominantly comprises NKG2A negative and single killer Ig-like receptor (KIR) positive NK cells.

Other embodiments will become apparent from a review of the ensuing detailed description, drawings and accompanying claims.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIG. 1A shows high viability of cells when co-cultured with DCOne mDC for 7 or 14 days without addition of cytokines. Cells were harvested and assessed for viability using Nucleo Counter NC-200.

FIG. 1B shows the frequency of NK cells on day 7 and 14 co-cultures of NK cells and DCOne mDC in a MLR assay. At day 7 and 14 cells were harvested and stained with anti-CD56, CD3, NKG2C and CD57 specific antibodies and analyzed by flow cytometry.

FIG. 1C shows induced production of immune-cell-recruiting chemokines, proinflammatory and effector cytokines when NK cells are co-cultured with DCOne cells. Supernatants from NK cells co-cultured in the presence or absence DCOne mDC was harvested on day 4. Multi-analyte profiling of cytokines and chemokines was performed using the Luminex MAGPIX® system (Luminex Corporation, USA). The levels of cytokines and chemokines were determined using magnetic antibody-coated beads (R&D). All analyses were performed according to the manufacturers' protocols. Acquired fluorescence data were analyzed by the 4.3×PONENT software (Luminex).

FIG. 2A shows the expansion of NK cells on day 14 co-cultures of NK cells and DCOne mDC, compared to cultures of NK cells alone, in the absence and/or presence of cytokines IL-2, IL-15, or a combination of IL-2 and IL-15 (IL-2/IL-15), IL-2 and IL-21 (IL-2/IL-15), or IL-15 and IL-21 (IL-15/IL-21). At day 14 cells were harvested and stained with anti-CD56, CD3, NKG2C and CD57 specific antibodies and analyzed by flow cytometry.

FIG. 2B is a set of images illustrating the flow cytometry analyses of NKG2C+/CD57+ NK cells at day 0 in CD56+/CD3− NK cells from one representative donor followed by 14 days expansion with IL-2 or IL-15, with or without DCOne derived mDCs, according to an experimental example. At day 14 of co-culturing, cells were harvested and stained with anti-CD56, CD3, NKG2C and CD57 specific antibodies and analyzed by flow cytometry.

FIG. 3 shows the expansion of memory (NKG2C+/CD57+) NK cells on day 14 co-cultures of NK cells and DCOne mDC, compared to NK cells cultured alone without DCOne mDC, in the absence and/or presence of cytokines IL-2, IL-15, or a combination of IL-2 and IL-15, IL-2 and IL-21, or IL-15 and IL-21. At day 14 cells were harvested and stained with anti-CD56, CD3, NKG2C and CD57 specific antibodies and analyzed by flow cytometry.

FIG. 4 shows the expansion of precursor memory (NKG2C+/CD57−) NK cells on day 14 co-cultures of NK cells and DCOne mDC, compared to NK cells cultured alone without DCOne mDC, in the absence and/or presence of cytokines IL-2, IL-15, or a combination of IL-2 and IL-15, IL-2 and IL-21, or IL-15 and IL-21. At day 14 cells were harvested and stained with anti-CD56, CD3, NKG2C and CD57 specific antibodies and analyzed by flow cytometry.

FIG. 5 shows the activation of NK cells co-cultured with DCOne mDCs for 14 days, compared to NK cells cultured alone without DCOne mDCs, in the absence and/or presence of cytokines IL-2, IL-15, or a combination of IL-2 and IL-15, IL-2 and IL-21, or IL-15 and IL-21. At day 7, cells were harvested and stained with anti-CD56, CD3, CD25 specific antibodies and analyzed by flow cytometry.

FIG. 6A depicts images showing flow cytometry analysis of the whole NK cell population and NKG2C+/CD57+ adaptive NK cells subpopulation after 14 days expansion with IL-15 and DCOne-derived mDCs according to one embodiment of the present disclosure.

FIG. 6B illustrates the percentage of FCεRIg⁻NKG2C⁺ cells from total NKG2C⁺ cells NK cells expanded 14 days using DCOne-derived mDC in the presence of cytokines IL-2, IL-15, or a combination of IL-2 and IL-15, IL-2 and IL-21, or IL-15 and IL-21.

FIG. 7 depicts graphs showing enhanced NK cell mediated cytotoxicity against anti-CD38 opsonized RAJI tumor cells according to one embodiment of the present disclosure.

FIGS. 8A-8D illustrate flow cytometry analysis of activating ligands known to be associated with expansion of adaptive NK cells ex vivo.

FIG. 9A-9C depict graphs showing enhanced chemokine and cytokine production produced by DCOne mDC stimulated NK cells after interaction with anti-CD38 opsonized RAJI tumor cells. FIG. 9A is a set of flow cytometry images illustrating increased DCOne mDC stimulated (in the presence of IL-2, IL-15, or a combination of IL-2 and IL-15, IL-2 and IL-21, or IL-15 and IL-21) IFNγ positive NK cells after interaction with or without anti-CD38 opsonized RAJI tumor cells. FIG. 9B is graphically illustrates increased DCOne mDC stimulated (in the presence of IL-2, IL-15, or a combination of IL-2 and IL-15, IL-2 and IL-21, or IL-15 and IL-21) IFNγ positive NKG2C negative conventional and NKG2C positive memory NK cells after interaction with or without anti-CD38 opsonized RAJI tumor cells. FIG. 9C depicts NK cells expanded with DCOne derived mDCs produce more CCL3, CCL4, GM-CSF, IFN-γ, and TNF-α upon tumor cell interaction opsonized with anti CD38 antibody.

FIGS. 10A and 10B illustrate tumor cell and NK cell persistence 3-days post NK cell-tumor co-culture. NK cells expanded with DCOne mDCs in the presence of cytokines persist in NK-tumor cell co-cultures resulting in higher lymphocyte to tumor cell ratio.

### DETAILED DESCRIPTION

Provided herein are methods for improving the stimulation and expansion of immune cells such as natural killer (NK) cells, as well as methods for generating memory NK cells. In certain embodiments, the methods comprise contacting a population of immune cells (e.g., comprising NK cells) with a modified cell of leukemic origin. Methods of treating a disease or disorder are also provided, comprising the administration of the expanded and activated NK cells into a subject. Such methods may increase the viability and enhance the activation of the NK cells. Such methods may also prolong the duration of the clinical effect of a genetically modified NK cell, and/or function to stabilize subjects following adoptive cell therapy. In certain embodiments, the modified cell of leukemic origin is non-proliferating (e.g., via irradiation). In certain embodiments, the non-proliferating modified cell of leukemic origin is a non-proliferating DCOne derived cell.

It is to be understood that the methods described herein are not limited to particular methods and experimental conditions disclosed herein as such methods and conditions may vary. It is also to be understood that the terminology used herein is for the purpose of describing particular embodiments only and is not intended to be limiting. The methods described herein use conventional molecular and cellular biological and immunological techniques that are well within the skill of the ordinary artisan. Such techniques are well known to the skilled artisan and are explained in the scientific literature.

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, e.g., ±5%, ±1%, or ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

The term “antigen” or “antigenic,” as used in relation to a polypeptide as described herein, refers generally to a biological molecule which contains at least one epitope specifically recognized by a T cell receptor, an antibody, or other elements of specific humoral and/or cellular immunity. The whole molecule may be recognized, or one or more portions of the molecule, for instance following intracellular processing of a polypeptide into an MHC peptide antigen complex and subsequent antigen presentation. The term “antigenic polypeptide” is interchangeable with “polypeptide antigen.” This terminology includes antigenic parts of said polypeptides, for instance produced after intracellular processing of a polypeptide and in the context of a MHC peptide antigen complex. The term “antigen” or “antigenic” includes reference to at least one, or more, antigenic epitopes of a polypeptide as described herein. In certain embodiments, a “non-tumor antigen” refers to herein as an antigen that is not derived from a tumor. For example, in certain embodiments, a non-tumor antigen may be a foreign antigen.

As used herein, the term “autologous” is meant to refer to any material derived from the same individual to which it is later to be re-introduced into the individual.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, an amount that when administered to a mammal, causes a detectable level of immune suppression or tolerance compared to the immune response detected in the absence of the composition of the disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “immune response,” as used herein, includes T cell mediated and/or B cell mediated immune responses. Exemplary immune functions of T cells include, e.g., cytokine production and induction of cytotoxicity in other cells. B cell functions include antibody production. In addition, the term includes immune responses that are indirectly affected by T cell activation, e.g., antibody production and activation of cytokine responsive cells, e.g., macrophages. Immune cells involved in the immune response include lymphocytes, such as B cells and T cells (CD4⁺ and CD8⁺ cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes. In certain embodiments, the term refers to a T cell mediated immune response. The immune response may in some embodiments be a T cell-dependent immune response. The skilled person understands that the phrase “immune response against a tumor” also includes immune responses against a non-human antigenic polypeptide that is introduced into the tumor micro-environment by intratumoral administration, such as intratumoral administration of (i) dendritic cells, including autologous or allogeneic dendritic cells, loaded with said polypeptide or (ii) viruses comprising a nucleic acid encoding said polypeptide.

The term “T cell dependent immune response,” as used herein, refers to an immune response wherein either T cells, B cells or both T cell and B cell populations are activated, and wherein T cells further assist T and B cells and other immune cells in executing their function.

The term “immunosuppressive” is used herein to refer to reducing overall immune response.

“Insertion/deletion,” commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, e.g., a human.

“Parenteral” administration of an immunogenic composition includes, e.g., subcutaneous (s.c.), intravenous (i.v.), intramuscular (i.m.), intradermal, intraperitoneal, or intrasternal injection, or infusion techniques.

The term “polynucleotide,” as used herein, is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species. For example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody. The term “subject,” as used herein, refers to the recipient of a method as described herein, i.e., a recipient that can mount a cellular immune response, and is a mammal. In certain embodiments, the subject is a human. In certain embodiments, the subject is a domesticated animal, e.g., a horse, a cow, a pig, a sheep, a dog, a cat, etc. The terms “patient” and “subject” may be used interchangeably. In certain embodiments, the subject is a human suffering from a tumor (e.g., a solid tumor) or an infectious disease. In certain embodiments, the subject is a domesticated animal suffering from a tumor (e.g., a solid tumor).

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The term “tumor,” as used herein, includes reference to cellular material, e.g., a tissue, proliferating at an abnormally high rate. A growth comprising neoplastic cells is a neoplasm, also known as a “tumor,” and generally forms a distinct tissue mass in a body of a subject. A tumor may show partial or total lack of structural organization and functional coordination with the normal tissue. As used herein, a tumor is intended to encompass hematopoietic tumors as well as solid tumors. In certain embodiments, the tumor is a solid tumor. The term “tumor,” as used herein, includes reference to the tumor micro-environment or tumor site, i.e., the area within the tumor and the area directly outside the tumorous tissue. In certain embodiments, the tumor micro-environment or tumor site includes an area within the boundaries of the tumor tissue. In certain embodiments, the tumor micro-environment or tumor site includes the tumor interstitial compartment of a tumor, which is defined herein as all that is interposed between the plasma membrane of neoplastic cells and the vascular wall of the newly formed neovessels. As used herein, the terms “tumor micro-environment” or “tumor site” refers to a location within a subject in which a tumor resides, including the area immediately surrounding the tumor.

A tumor may be benign (e.g., a benign tumor) or malignant (e.g., a malignant tumor or cancer). Malignant tumors can be broadly classified into three major types: those arising from epithelial structures are called carcinomas, those that originate from connective tissues such as muscle, cartilage, fat or bone are called sarcomas, and those affecting hematopoietic structures (structures pertaining to the formation of blood cells) including components of the immune system, are called leukemias and lymphomas. Other tumors include, but are not limited to, neurofibromatosis. In certain exemplary embodiments, the tumor is a glioblastoma. In certain exemplary embodiments, the tumor is an ovarian cancer (e.g., an epithelial ovarian cancer, which can be further subtyped into a serous, a clear cell, an endometrioid, a mucinous, or a mixed epithelial ovarian cancer).

Solid tumors are abnormal masses of tissue that can be benign or malignant. In certain embodiments, solid tumors are named for the type of cells that form them (such as sarcomas, carcinomas, and lymphomas). Examples of solid tumors, such as sarcomas and carcinomas, include, but are not limited to, liposarcoma, fibrosarcoma, chondrosarcoma, osteosarcoma, myxosarcoma, and other sarcomas, mesothelioma, synovioma, leiomyosarcoma, Ewing's tumor, colon carcinoma, rhabdomyosarcoma, pancreatic cancer, lymphoid malignancy, lung cancers, breast cancer, prostate cancer, ovarian cancer, hepatocellular carcinoma, adenocarcinoma, basal cell carcinoma, sweat gland carcinoma, squamous cell carcinoma, medullary thyroid carcinoma, pheochromocytomas sebaceous gland carcinoma, papillary thyroid carcinoma, papillary adenocarcinomas, papillary carcinoma, medullary carcinoma, bronchogenic carcinoma, hepatoma, renal cell carcinoma, bile duct carcinoma, Wilms' tumor, choriocarcinoma, cervical cancer, seminoma, testicular tumor, bladder carcinoma, melanoma, CNS tumors (e.g., a glioma, e.g., brainstem glioma and mixed gliomas, glioblastoma (e.g., glioblastoma multiforme), germinoma, astrocytoma, craniopharyngioma, medulloblastoma, ependymoma, Schwannoma, CNS lymphoma, acoustic neuroma, pinealoma, hemangioblastoma, meningioma, oligodendroglioma, retinoblastoma, neuroblastoma, and brain metastases), and the like.

Carcinomas that can be amenable to therapy by a method disclosed herein include, but are not limited to, squamous cell carcinoma (various tissues), basal cell carcinoma (a form of skin cancer), esophageal carcinoma, bladder carcinoma, including transitional cell carcinoma (a malignant neoplasm of the bladder), hepatocellular carcinoma, colorectal carcinoma, bronchogenic carcinoma, lung carcinoma, including small cell carcinoma and non-small cell carcinoma of the lung, colon carcinoma, thyroid carcinoma, gastric carcinoma, breast carcinoma, ovarian carcinoma, adrenocortical carcinoma, pancreatic carcinoma, sweat gland carcinoma, prostate carcinoma, papillary carcinoma, adenocarcinoma, sebaceous gland carcinoma, medullary carcinoma, papillary adenocarcinoma, ductal carcinoma in situ or bile duct carcinoma, cystadenocarcinoma, renal cell carcinoma, choriocarcinoma, Wilm's tumor, seminoma, embryonal carcinoma, cervical carcinoma, testicular carcinoma, nasopharyngeal carcinoma, osteogenic carcinoma, epithelial carcinoma, uterine carcinoma, and the like.

Sarcomas that can be amenable to therapy by a method disclosed herein include, but are not limited to, myxosarcoma, chondrosarcoma, chordoma, osteogenic sarcoma, liposarcoma, fibrosarcoma, angiosarcoma, lymphangiosarcoma, endotheliosarcoma, osteosarcoma, mesothelioma, Ewing's sarcoma, leiomyosarcoma, rhabdomyosarcoma, lymphangioendotheliosarcoma, synovioma, and other soft tissue sarcomas.

The term “modified cell of leukemic origin,” as used herein, refers to a cell that can take up an antigen such as an antigenic polypeptide into its cell, and presents the antigen, or an immunogenic part thereof together with an MHC class I complex or MHC class II complex. These cells are distinct from modified K562 cells and other artificial Antigen Presenting Cells (aAPCs), as described for example, in WO2005/118788, By contrast, an aAPC is engineered to express at least one stimulatory ligand and at least one costimulatory ligand where the ligands each specifically bind with a cognate molecule on a T cell of interest, thereby mediating expansion of the T cell. By contrast, and without being bound to any particular theory, a modified cell of leukemic origin stimulates NK cells and natively expresses a stimulatory ligand and a costimulatory ligand where the ligands each specifically bind with a cognate molecule on the NK cell. In certain embodiments, the modified cell of leukemic origin is a cell derived from cell line DCOne as deposited under the conditions of the Budapest treaty with the DSMZ under accession number DSMZ ACC3189 on 15 Nov. 2012. The process of obtaining mature cells from the deposited DCOne cell line is, for instance, described in EP2931878B1.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Modified Cell of Leukemic Origin

Provided herein are methods comprising the use of a modified cell of leukemic origin to stimulate and expand immune cells, generate antigen-specific immune cells, and for methods of treatment. As used herein, the term “modified cell of leukemic origin” refers to a cell capable of taking up an antigen such as an antigenic polypeptide, and capable of presenting the antigen, or an immunogenic part thereof, together with an MHC class I complex or MHC class II complex. A modified cell of leukemic origin provided herein comprises a mature dendritic cell phenotype. The term “dendritic cell,” as used herein, refers to a professional antigen presenting cell (APC) that can take up an antigen such as an antigenic polypeptide into its cell, and presents the antigen, or an immunogenic part thereof together with an MHC class I complex or MHC class II complex. Having a mature dendritic cell phenotype means that the modified cell of leukemic origin is capable of performing similar functions to those of a mature dendritic cell. The term includes both immature dendritic cells (“imDC”) and mature dendritic cells (“mDC”), depending on maturity.

In certain embodiments, the modified cell of leukemic origin is derived from leukemia cells. In certain embodiments, the modified cell of leukemic origin is derived from a patient having leukemia. In certain embodiments, the modified cell of leukemic origin is derived from the peripheral blood of a patient having leukemia. In certain embodiments, the modified cell of leukemic origin is derived from the peripheral blood of a patient having acute myeloid leukemia. The skilled artisan will recognize that a modified cell of leukemic origin can be derived from any patient obtained peripheral blood, wherein the patient has any type of leukemia, given that the modified cell of leukemic origin thus derived comprises the characteristics disclosed herein.

In certain embodiments, the modified cell of leukemic origin is CD34-positive, CD1a-positive, and CD83-positive. In certain embodiments, the modified cell of leukemic origin comprises a cell surface marker selected from the group consisting of CD14, DC-SIGN, Langerin, CD40, CD70, CD80, CD83, CD86, and any combination thereof. In certain embodiments, the modified cell of leukemic origin comprises an MHC class I molecule. In certain embodiments, the modified cell of leukemic origin comprises an MHC class II molecule. In certain embodiments, the modified cell of leukemic origin is CD34-positive, CD1a-positive, CD83-positive, and CD14-negative. In certain embodiments, the modified cell of leukemic origin is CD40-positive, CD80-positive, and CD86-positive. In certain embodiments, the modified cell of leukemic origin is CD34-positive, CD1a-positive, CD83-positive, CD40-positive, CD80-positive, CD86-positive, and CD14-negative.

In certain embodiments, the modified cell of leukemic origin comprises a genetic aberration between chromosome 11p15.5 to 11p12. In certain embodiments, the genetic aberration encompasses about 16 Mb of genomic regions (e.g., from about 20.7 Mb to about 36.6 Mb). In certain embodiments, the genetic aberration contains a loss of about 60 known and unknown genes.

In certain embodiments, the modified cell of leukemic origin comprises a co-stimulatory molecule. In certain embodiments, the co-stimulatory molecule includes, without limitation, an MHC class I molecule, B and T lymphocyte attenuator (BTLA), and Toll ligand receptor. Examples of co-stimulatory molecules include CD112, CD155, CD70, CD80, CD86, 4-1BBL (CD137-ligand), OX40L, CD30L, CD40, PD-L1, ICOSL, ICAM-1, lymphocyte function-associated antigen 3 (LFA3 (CD58)), K12/SECTM1, LIGHT, HLA-E, B7-H3 and CD83. In certain embodiments, the co-stimulatory molecule is selected from CD112, CD155, and/or CD58.

In certain embodiments, the modified cell of leukemic origin comprises at least one endogenous antigen. Depending on the leukemic origin of the modified cell, the modified cell of leukemic origin may comprise at least one known endogenous antigen that is specific to the leukemic origin. In certain embodiments, the endogenous antigen is a tumor-associated antigen. In certain embodiments, an endogenous tumor-associated antigen may be selected from the group consisting of WT-1, RHAMM, PRAME, p53, Survivin, and MUC-1.

In certain embodiments, the modified cell of leukemic origin comprises an exogenous antigen or peptide fragments thereof. Such an exogenous antigen may be provided to the modified cell of leukemic origin via various antigen loading strategies. For example, strategies for loading a modified cell of leukemic origin may include, without limitation, the use of synthetic long peptides, mRNA loading, peptide-pulsing, protein-loading, tumor lysate-loading, coculturing with a tumor cell, RNA/DNA transfection or viral transduction. Other strategies for loading a modified cell of leukemic origin are known to those of skill in the art and may be used to load a modified cell of leukemic origin with an exogenous antigen. In general, the modified cell of leukemic origin will process the exogenous antigen via particular molecules, e.g., via MHC I or MHC II. As such, an exogenous antigen comprised by the modified cell of leukemic origin may be an MHC class I antigen or an MHC class II antigen. In certain embodiments, the exogenous antigen is a tumor-associated antigen. For example, in certain embodiments, the modified cell of leukemic origin is loaded with NY-ESO-1 peptide and/or WT-1 peptide, or a tumor-independent antigen such as CMVpp65. In certain embodiments, the exogenous antigen is associated with a disease or disorder, e.g., a non-cancer-associated disease or disorder. It will be appreciated by those of ordinary skill in the art that any tumor-associated antigen or antigen associated with a disease or disorder can be provided to the modified cell of leukemic origin described herein. As such, in certain embodiments, a modified cell of leukemic origin comprises any tumor-associated antigen or antigen associated with a disease or disorder contemplated by those skilled in the art.

Loading of the modified cell of leukemic origin with an exogenous antigen or peptide fragments thereof may be performed at any time. The skilled person will be able to determine and carry out the specific timing of loading of the modified cell of leukemic origin to best suit their needs. For example, in certain embodiments, the modified cell of leukemic origin is loaded with an exogenous antigen or peptide fragments thereof prior to its exhibiting a mature dendritic cell phenotype. In certain embodiments, the modified cell of leukemic origin is loaded with the exogenous antigen or peptide fragments thereof during transition of the modified cell of leukemic origin to a mature dendritic cell phenotype. In certain embodiments, the modified cell of leukemic origin is loaded with the exogenous antigen or peptide fragments thereof after the modified cell of leukemic origin exhibits a mature dendritic cell phenotype.

In certain embodiments, the modified cell of leukemic origin is a cell of cell line DCOne as described in PCT Publication Nos. WO 2014/006058 and WO 2014/090795, the disclosures of which are incorporated by reference herein in their entireties. In certain embodiments, modified cell of leukemic origin is a cell of cell line DCOne and comprises a mature dendritic cell phenotype that is CD34-positive, CD1a-positive, and CD83-positive. In certain embodiments, modified cell of leukemic origin is a cell of cell line DCOne and is CD34-positive, CD1a-positive, and CD83-positive. In certain embodiments, modified cell of leukemic origin is a cell of cell line DCOne and comprises a cell surface marker selected from the group consisting of CD14, DC-SIGN, Langerin, CD80, CD86, CD40, CD70, and any combination thereof. In certain embodiments, modified cell of leukemic origin is a cell of cell line DCOne and comprises MHC class I. In certain embodiments, modified cell of leukemic origin is a cell of cell line DCOne and comprises MHC class II. In certain embodiments, the modified cell of leukemic origin is a cell of cell line DCOne and is CD34-positive, CD1a-positive, CD83-positive, and CD14-negative. In certain embodiments, the modified cell of leukemic origin is a cell of cell line DCOne and is CD40-positive, CD80-positive, and CD86-positive. In certain embodiments, the modified cell of leukemic origin is a cell of cell line DCOne and is CD34-positive, CD1a-positive, CD83-positive, CD40-positive, CD80-positive, CD86-positive, and CD14-negative. In certain embodiments, modified cell of leukemic origin is a cell of cell line DCOne and comprises a genetic aberration between chromosome 11p15.5 to 11p12. In certain embodiments, modified cell of leukemic origin is a cell of cell line DCOne and comprises a genetic aberration that encompasses about 16 Mb of genomic regions (e.g., from about 20.7 Mb to about 36.6 Mb). In certain embodiments, modified cell of leukemic origin is a cell of cell line DCOne and comprises a genetic aberration that contains a loss of about 60 known and unknown genes.

In certain embodiments, the modified cell of leukemic origin is a plasmacytoid dendritic human cell lines (pDC) as described in U.S. Pat. Nos. 7,341,870 and 9,783,782, which are specifically incorporated herein by reference. In particular embodiments, the plasmacytoid dendritic human cell line is the cell line designated GEN2.2 which is deposited in CNCM (Collection Nationale de Cultures de Microorganismes [National Collection of Cultures of Microorganisms], Pasteur Institute, 25 rue du Docteur Roux, F-75015 Paris) under number CNCM 1-2938. In other embodiments, the plasmacytoid dendritic human cell line is the cell line designated GEN 3, which with deposited with the CNCM under the CNCM number 1-3110.

As provided herein, certain methods are directed to the use of a modified cell of leukemic origin, wherein the modified cell is non-proliferating. In certain embodiments, the modified cell of leukemic origin has been irradiated. In certain embodiments, the modified cell of leukemic origin has been irradiated prior to its use in a method disclosed herein. Irradiation can, for example, be achieved by gamma irradiation at 30-150 Gy, e.g., 100 Gy, for a period of 1 to 3 hours, using a standard irradiation device (Gammacell or equivalent). Irradiation ensures that any remaining progenitor cell in a composition comprising the modified cell of leukemic origin, e.g., a CD34 positive cell, cannot continue dividing. The cells may, for example, be irradiated prior to injection into patients, when used as a vaccine, or immediately after cultivating is stopped. In certain embodiments, the cells are irradiated to inhibit their capacity to proliferate and/or expand, while maintaining their immune stimulatory capacity.

### C. Sources of NK Cells

Prior to expansion, a source of NK cells is obtained from a subject for ex vivo manipulation. Sources of NK cells for ex vivo manipulation may also include, e.g., autologous or heterologous donor blood, cord blood, or bone marrow. For example, the source of NK cells may be from the subject to be treated with the modified immune cells of the disclosure, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

NK cells can be obtained from a number of sources, including blood, peripheral blood mononuclear cells, bone marrow, lymph node tissue, spleen tissue, umbilical cord, lymph, or lymphoid organs. In certain embodiments, the NK cells are human cells. With reference to the subject to be treated, the NK cells may be allogeneic and/or autologous. The NK cells typically are primary cells, such as those isolated directly from a subject and/or isolated from a subject and frozen.

In certain embodiments, the methods include isolating NK cells from the subject, preparing, processing, culturing, and/or engineering them. In certain embodiments, preparation of the NK cells includes one or more culture and/or preparation steps. The NK cells may be isolated from a sample, such as a biological sample, e.g., one obtained from or derived from a subject. In certain embodiments, the subject from which the NK cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the NK cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain embodiments, the sample from which the NK cells are derived or isolated is blood or a blood-derived sample, or is derived from an apheresis or leukapheresis product.

Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In certain embodiments, the NK cells are derived from cell lines, e.g., NK cell lines. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In certain embodiments, NK cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In certain embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In certain embodiments, a washing step is accomplished by tangential flow filtration (TFF) according to the manufacturers instructions. In certain embodiments, the cells are resuspended in a variety of biocompatible buffers after washing. In certain embodiments, components of a blood cell sample are removed and the cells directly resuspended in culture media. In certain embodiments, the methods include density-based cell separation methods, such as the preparation of white blood cells from peripheral blood by lysing the red blood cells and centrifugation through a Percoll or Ficoll gradient.

In certain embodiments, NK cells are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In certain embodiments, the isolation methods include the separation of different cell types based on the expression or presence in the cell of one or more specific molecules, such as surface markers, e.g., surface proteins, intracellular markers, or nucleic acid. In certain embodiments, any known method for separation based on such markers may be used. In certain embodiments, the separation is affinity- or immunoaffinity-based separation. For example, the isolation in certain embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In certain embodiments, both fractions are retained for further use. In certain embodiments, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain embodiments, multiple rounds of separation steps are carried out, where the positively or negatively selected fraction from one step is subjected to another separation step, such as a subsequent positive or negative selection. In certain embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In certain embodiments, one or more of the NK cell populations is enriched for or depleted of cells that are positive for (marker+) or express high levels (marker^{high}) of one or more particular markers, such as surface markers, or that are negative for (marker−) or express relatively low levels (marker^{low}) of one or more markers. For example, in certain embodiments, specific subpopulations of NK cells, such as memory NK cells may be selected. Such subpopulation of NK cells positive for expressing high levels of one or more surface markers, e.g., NKG2C-positive and/or CD57-positive NK cells, are isolated by positive or negative selection techniques. In certain embodiments, such markers are those that are absent or expressed at relatively low levels on certain populations of NK cells (such as non-memory NK cells) but are present or expressed at relatively higher levels on certain other populations of NK cells (such as memory NK cells). In one embodiment, the NK cells are enriched for (i.e., positively selected for) cells that are positive or expressing high surface levels of NKG2C and/or CD57 and depleted of (e.g., negatively selected for) cells that do not express NKG2C and/or CD57. In certain embodiments, the NK cells are enriched for memory NK cells positive for both NKG2C and for CD57. For example, NKG2C+, CD57+ NK cells can be positively selected using anti-marker conjugated magnetic beads (e.g., DYNABEADS®).

In certain exemplary embodiments, the NK cells are low FcεRIγ expression memory NK cells. In certain exemplary embodiments, the expanded population of activated NK cells predominantly comprises FcεRIγ negative memory NK cells, i.e., the percentage or frequency of FcεRIγ expression is low in the total NK cells. In certain embodiments, the NK cells are FcεRIγ negative memory NK cells.

In certain embodiments, the cells are incubated and/or cultured prior to or in connection with genetic engineering. The incubation steps can include culture, cultivation, stimulation, activation, and/or propagation. In certain embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In certain exemplary embodiments, a stimulatory agent may be chosen from growth factors. In certain exemplary embodiments, the population of the immune cells is co-cultured in the presence of at least one growth factor, optionally, wherein the at least one growth factor is selected from serum, insulin, IFN□, interleukin-2 (IL-2), IL-4, IL-7, IL-12, IL-15, IL-18, IL-21, GM-CSF, TNF-α, or any combination thereof. In certain embodiments, the stimulatory agent is chosen from cytokines. In certain embodiments, the stimulating agents include IL-2, IL-7, IL-15 and/or IL-21. For example, an IL-2 concentration of at least about 10 units/mL. In certain exemplary embodiments, the concentration of IL-2 in the co-culture is about 10 IU/ml-6000 IU/mL, about 50 IU/ml-200 IU/mL, or about 100 IU/mL. In certain exemplary embodiments, the concentration of IL-15 in the co-culture is about 5 ng/mL-150 ng/mL, about 50 ng/ml-150 ng/milord about 150 ng/mL. In certain exemplary embodiments, the concentration of IL-21 in the co-culture is about 5 ng/mL-150 ng/mL, about 50 ng/ml-150 ng/milord about 150 ng/mL. Enrichment of a NK cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in certain embodiments, a concentration of 2 billion cells/mi is used. In one embodiment, a concentration of 1 billion cells/mi is used. In a further embodiment, greater than 100 million cells/mi is used. In a further embodiment, a concentration of cells of 10, 15, 20, 25, 30, 35, 40, 45, or 50 million cells/mi is used. In yet another embodiment, a concentration of cells from 75, 80, 85, 90, 95, or 100 million cells/mi is used. In further embodiments, concentrations of 125 or 150 million cells/mi can be used. Using high concentrations can result in increased cell yield, cell activation, and cell expansion.

NK cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

### D. CAR-NK Cells

In certain embodiment, the disclosure employs modified NK cells comprising an immune receptor, wherein the immune receptor is a chimeric antigen receptor (CAR). Thus, in certain embodiments, the NK cell has been genetically modified to express the CAR. NK-CARs of the present disclosure are NK cells that comprise an antigen binding domain, a transmembrane domain, and an intracellular domain.

The antigen binding domain may be operably linked to another domain of the CAR, such as the transmembrane domain or the intracellular domain, both described elsewhere herein, for expression in the cell. In certain embodiments, a first nucleic acid sequence encoding the antigen binding domain is operably linked to a second nucleic acid encoding a transmembrane domain, and further operably linked to a third a nucleic acid sequence encoding an intracellular domain. The antigen binding domains described herein can be combined with any of the transmembrane domains described herein, any of the intracellular domains or cytoplasmic domains described herein, or any of the other domains described herein that may be included in a CAR. In certain embodiments, a CAR may also include a hinge domain as described herein. In certain embodiments, a CAR may also include a spacer domain as described herein. In certain embodiments, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In certain embodiments, the CAR has affinity to a target antigen on a target cell. The target antigen may include any type of protein, or epitope thereof, associated with the target cell. For example, the CAR may have affinity to a target antigen on a target cell that indicates a particular disease state of the target cell.

Depending on the desired antigen to be targeted, the CAR can be engineered to include an appropriate antigen binding domain that is specific to the desired antigen target. In certain embodiments, such an antigen can be introduced into a tumor cell, e.g., via a tumor-marking step as described herein. In certain embodiments, the target cell antigen is a tumor-associated antigen (TAA). In certain embodiments, the target cell antigen is a non-tumor-associated antigen (non-TAA, e.g., a tumor independent antigen). A CAR having specificity for any target antigen is suitable for use in a method as provided herein. In certain embodiments, the antigen that the CAR is specific for is matched to an antigen expressed by a tumor cell.

In certain embodiments, the immune receptor (e.g., CAR) provides specificity to the immune cell towards a target antigen. In certain embodiments, the CAR provided target antigen specificity is the same as the target antigen that the immune cell is specific for. In such embodiments, the CAR specificity is said to be matched with the endogenous specificity of the immune cell. In certain embodiments, the CAR-provided target antigen specificity is different than the target antigen for which the immune cell is specific. In such embodiments, the CAR specificity is said to be unmatched with the endogenous specificity of the immune cell. As such, a CAR having unmatched specificity with the endogenous specificity of the immune cell gives rise to a multi-specific (e.g., a bispecific) immune cell.

As described herein, a CAR having affinity for a specific target antigen on a target cell may comprise a target-specific binding domain. In certain embodiments, the target-specific binding domain is a murine target-specific binding domain, e.g., the target-specific binding domain is of murine origin. In certain embodiments, the target-specific binding domain is a human target-specific binding domain, e.g., the target-specific binding domain is of human origin.

In certain embodiments, a CAR may have affinity for one or more target antigens on one or more target cells. In certain embodiments, a CAR may have affinity for one or more target antigens on a target cell. In such embodiments, the CAR is a bispecific CAR, or a multi-specific CAR. In certain embodiments, the CAR comprises one or more target-specific binding domains that confer affinity for one or more target antigens. In certain embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo linker or a polypeptide linker, an Fc hinge region, or a membrane hinge region.

In certain embodiments, the antigen binding domain is selected from the group consisting of an antibody, an antigen binding fragment (Fab), and a single-chain variable fragment (scFv). The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen binding domain portion comprises a mammalian antibody or a fragment thereof. The choice of antigen binding domain may depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In certain embodiments, the antigen binding domain (e.g., PSCA binding domain) comprises an scFv having the configuration from N-terminus to C-terminus, VH-linker-VL. In certain embodiments, the antigen binding domain comprises an scFv having the configuration from N-terminus to C-terminus, VL-linker-VH. Those of skill in the art would be able to select the appropriate configuration for use in the present disclosure.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers. Those of skill in the art would be able to select the appropriate linker sequence for use in the present disclosure. In certain embodiments, an antigen binding domain of the present disclosure comprises a heavy chain variable region (VH) and a light chain variable region (VL), wherein the VH and VL is separated by a GS linker sequence.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Immunol 2009 183(4):2277-85; Giomarelli et al., Thromb Haemost 2007 97(6):955-63; Fife eta., J Clin lnvst 2006 116(8):2252-61; Brooks et al., Immunotechnology 1997 3(3):173-84; Moosmayer et al., Ther Immunol 1995 2(10:31-40). Agonistic scFvs having stimulatory activity have been described (see, e.g., Peter et al., J Bio Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In certain embodiments, the antigen binding domain may be derived from the same species in which the immune cell may be administered to. For example, for use in humans, the antigen binding domain of the CAR may comprise a human antibody or a fragment thereof. In certain embodiments, the antigen binding domain may be derived from a different species in which the immune cell may be administered to. For example, for use in humans, the antigen binding domain of the CAR may comprise a murine antibody or a fragment thereof.

### Transmembrane Domain

A CAR may comprise a transmembrane domain that connects the antigen binding domain of the CAR to the intracellular domain of the CAR. The transmembrane domain of a CAR is a region that is capable of spanning the plasma membrane of a cell (e.g., an immune cell or precursor thereof). The transmembrane domain is for insertion into a cell membrane, e.g., a eukaryotic cell membrane. In certain embodiments, the transmembrane domain is interposed between the antigen binding domain and the intracellular domain of a CAR.

In certain embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this disclosure include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In certain embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a CAR.

In certain embodiments, the transmembrane domain further comprises a hinge region. In certain embodiments, a CAR may also include a hinge region. The hinge region of the CAR is a hydrophilic region which is located between the antigen binding domain and the transmembrane domain. In certain embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In certain embodiments, a CAR includes a hinge region that connects the antigen binding domain with the transmembrane domain, which, in turn, connects to the intracellular domain. The hinge region is typically capable of supporting the antigen binding domain to recognize and bind to the target antigen on the target cells (see, e.g., Hudecek et al., Cancer Immunol. Res. (2015) 3(2): 125-135). In certain embodiments, the hinge region is a flexible domain, thus allowing the antigen binding domain to have a structure to optimally recognize the specific structure and density of the target antigens on a cell such as tumor cell. Id. The flexibility of the hinge region permits the hinge region to adopt many different conformations.

In certain embodiments, the hinge region is an immunoglobulin heavy chain hinge region. In certain embodiments, the hinge region is a hinge region polypeptide derived from a receptor (e.g., a CD8-derived hinge region).

The hinge region can have a length of from about 4 amino acids (aa) to about 50 amino acids (aa), e.g., from about 4 aa to about 10 aa, from about 10 aa to about 15 aa, from about aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142).

In certain embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. See, e.g., Yan et al., J. Biol. Chem. (2012) 287: 5891-5897.

### Intracellular Signaling Domain

A CAR also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the disclosure include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In certain embodiments, the intracellular signaling domain may be human CD3 zeta chain, FcγRIIII, FcsRI, cytoplasmic tails of Fc receptors, an immunoreceptor tyrosine-based activation motif (ITAM) bearing cytoplasmic receptors, and combinations thereof.

In certain embodiments, the intracellular signaling domain of the CAR includes any portion of one or more co-stimulatory molecules, such as at least one signaling domain from CD2, CD3, CD8, CD27, CD28, ICOS, 4-166, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon Rib), CD79a, CD79b, Fc□RIIa, DAP10, DAP12, T cell receptor (TCR), CD8, CD27, CD28, 4-166 (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, CD49D, ITGA6, VLA-6, CD49f, ITGAD, CD11d, ITGAE, CD103, ITGAL, CD11a, LFA-1, ITGAM, CDlib, ITGAX, CD11c, ITGBI, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In certain embodiments, the intracellular signaling domain includes at least one (e.g., one, two, three, four, five, six, etc.) ITAM motifs as described below. In certain embodiments, the intracellular signaling domain includes DAP10/CD28 type signaling chains. In certain embodiments, the intracellular signaling domain is not covalently attached to the membrane bound CAR, but is instead diffused in the cytoplasm.

Intracellular signaling domains suitable for use in a subject CAR of the present disclosure include immunoreceptor tyrosine-based activation motif (ITAM)-containing intracellular signaling polypeptides. In certain embodiments, an ITAM motif is repeated twice in an intracellular signaling domain, where the first and second instances of the ITAM motif are separated from one another by 6 to 8 amino acids. In certain embodiments, the intracellular signaling domain of a subject CAR comprises 3 ITAM motifs.

In certain embodiments, intracellular signaling domains includes the signaling domains of human immunoglobulin receptors that contain immunoreceptor tyrosine based activation motifs (ITAMs) such as, but not limited to, FcγRI, FcγRIIA, FcγRIIC, FcγRIIIA, and FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCεR1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In certain embodiments, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In certain embodiments, the intracellular signaling domain is derived from FCεR1G (also known as FCRG; Fc epsilon receptor I gamma chain; Fc receptor gamma-chain; fc-epsilon RI-gamma; fcRγ; fceRIγ; high affinity immunoglobulin epsilon receptor subunit gamma; immunoglobulin E receptor, high affinity, gamma chain; etc.). In certain embodiments, the intracellular signaling domain is derived from T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.). In certain embodiments, the intracellular signaling domain is derived from T cell surface glycoprotein CD3 epsilon chain (also known as CD3e, T cell surface antigen T3/Leu-4 epsilon chain, T cell surface glycoprotein CD3 epsilon chain, A1504783, CD3, CD3epsilon, T3e, etc.). In certain embodiments, the intracellular signaling domain is derived from T cell surface glycoprotein CD3 gamma chain (also known as CD3G, T cell receptor T3 gamma chain, CD3-GAMMA, T3G, gamma polypeptide (TiT3 complex), etc.). In certain embodiments, the intracellular signaling domain is derived from T cell surface glycoprotein CD3 zeta chain (also known as CD3Z, T cell receptor T3 zeta chain, CD247, CD3-ZETA, CD3H, CD3Q, T3Z, TCRZ, etc.). In certain embodiments, the intracellular signaling domain is derived from CD79A (also known as B cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In certain embodiments, an intracellular signaling domain suitable for use in an FN3 CAR of the present disclosure includes a DAP10/CD28 type signaling chain. In certain embodiments, an intracellular signaling domain suitable for use in an FN3 CAR of the present disclosure includes a ZAP70 polypeptide. In certain embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In certain embodiments, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

### E. TCR-NK Cells

In certain embodiments, the disclosure employs modified NK cells comprising a recombinant T cell receptor (TCR). Typically, NK cell-mediated recognition of the target cell is independent of MHC-I. T cell receptors (TCRs) are T cell-specific cell surface receptors that play a major role in the recognition of MHC-I-expressing target cells by T cells. Therefore, in certain embodiments, engineering NK cells with TCRs enables the recognition of MHCs by NK cells and would potentially improve the tumor-recognition potential of the NK cells. TCR-NK cells are described, for example, in Morton et al., “T cell receptor engineering of primary NK cells to therapeutically target tumors and tumor immune evasion”, J. Immunother. Cancer, 2022).

In certain embodiments, the NK cells have been genetically modified to express the TCR. Provided herein are modified NK cells comprising an immune receptor, wherein the immune receptor is a T cell receptor (TCR), e.g., an exogenous TCR. Thus, in some embodiments, the cell has been altered to contain specific T cell receptor (TCR) genes (e.g., a nucleic acid encoding an alpha/beta TCR). TCRs or antigen-binding portions thereof include those that recognize a peptide epitope or T cell epitope of a target polypeptide, such as an antigen of a tumor, viral or autoimmune protein. In certain embodiments, the TCR has binding specificity for a non-tumor-associated antigen. In certain embodiments, the TCR has binding specificity for a tumor-associated antigen (TAA). In certain embodiments, the antigen that the TCR is specific for, or is matched to, an antigen comprised by a tumor cell.

A TCR is a disulfide-linked heterodimeric protein comprised of six different membrane bound chains that participate in the activation of immune cells (e.g., T cells) in response to an antigen. Alpha/beta TCRs and gamma/delta TCRs are known. An alpha/beta TCR comprises a TCR alpha chain and a TCR beta chain. T cells expressing a TCR comprising a TCR alpha chain and a TCR beta chain are commonly referred to as alpha/beta T cells. Gamma/delta TCRs comprise a TCR gamma chain and a TCR delta chain. T cells expressing a TCR comprising a TCR gamma chain and a TCR delta chain are commonly referred to as gamma/delta T cells.

The TCR alpha chain and the TCR beta chain are each comprised of two extracellular domains, a variable region and a constant region. The TCR alpha chain variable region and the TCR beta chain variable region are required for the affinity of a TCR to a target antigen (e.g., a TAA, or non-tumor-associated antigen). Each variable region comprises three hypervariable or complementarity-determining regions (CDRs) which provide for binding to a target antigen. The constant region of the TCR alpha chain and the constant region of the TCR beta chain are proximal to the cell membrane. A TCR further comprises a transmembrane region and a short cytoplasmic tail. CD3 molecules are assembled together with the TCR heterodimer. CD3 molecules comprise a characteristic sequence motif for tyrosine phosphorylation, known as immunoreceptor tyrosine-based activation motifs (ITAMs). Proximal signaling events are mediated through the CD3 molecules, and accordingly, TCR-CD3 complex interaction plays an important role in mediating cell recognition events.

Stimulation of TCR is triggered by major histocompatibility complex molecules (MHCs) on antigen presenting cells that present antigen peptides to T cells and interact with TCRs to induce a series of intracellular signaling cascades. Engagement of the TCR initiates both positive and negative signaling cascades that result in cellular proliferation, cytokine production, and/or activation-induced cell death.

A TCR can be a wild-type TCR, a high affinity TCR, and/or a chimeric TCR. A high affinity TCR may be the result of modifications to a wild-type TCR that confers a higher affinity for a target antigen compared to the wild-type TCR. A high affinity TCR may be an affinity-matured TCR. In certain embodiments, it may be desired to obtain a TCR of lower affinity as compared to the wild-type TCR. Such lower affinity TCRs may also be referred to as affinity-tuned TCRs. Methods for modifying TCRs and/or the affinity-maturation/affinity-tuning of TCRs are known to those of skill in the art. Techniques for engineering and expressing TCRs include, but are not limited to, the production of TCR heterodimers which include the native disulfide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840).

In certain embodiments, the exogenous TCR is a full TCR or an antigen-binding fragment thereof. In certain embodiments, the TCR is an intact or full-length TCR, including TCRs in the αβ form or γδ form. In certain embodiments, the TCR is an antigen-binding portion that is less than a full-length TCR but that binds to a specific peptide bound in an MHC molecule, such as binds to an MHC-peptide complex. In certain embodiments, an antigen-binding portion or fragment of a TCR can contain only a portion of the structural domains of a full-length or intact TCR, but yet is able to bind the peptide epitope, such as an MHC-peptide complex, to which the full TCR binds. In certain embodiments, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

In certain embodiments, the variable domains of the TCR contain hypervariable loops, or CDRs, which generally are the primary contributors to antigen recognition and binding capabilities and specificity. In certain embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al, Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, CDR3 is the main CDR responsible for antigen binding or specificity, or is the most important among the three CDRs on a given TCR variable region for antigen recognition, and/or for interaction with the processed peptide portion of the peptide-MHC complex. In certain embodiments, the CDR1 of the alpha chain can interact with the N-terminal part of certain antigenic peptides. In certain embodiments, CDR1 of the beta chain can interact with the C-terminal part of the peptide. In certain embodiments, CDR2 contributes most strongly to or is the primary CDR responsible for the interaction with or recognition of the MHC portion of the MHC-peptide complex. In certain embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In certain embodiments, a TCR contains a variable alpha domain (V_{α}) and/or a variable beta domain (V_{β}) or antigen-binding fragments thereof. In certain embodiments, the α-chain and/or β-chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33, 1997). In certain embodiments, the α chain constant domain is encoded by the TRAC gene (IMGT nomenclature) or is a variant thereof. In certain embodiments, the β chain constant region is encoded by TRBC1 or TRBC2 genes (IMGT nomenclature) or is a variant thereof. In certain embodiments, the constant domain is adjacent to the cell membrane. For example, in certain embodiments, the extracellular portion of the TCR formed by the two chains contains two membrane-proximal constant domains, and two membrane-distal variable domains, which variable domains each contain CDRs.

It is within the level of a skilled artisan to determine or identify the various domains or regions of a TCR. In certain embodiments, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). The IMGT numbering system should not be construed as limiting in any way, as there are other numbering systems known to those of skill in the art, and it is within the level of the skilled artisan to use any of the numbering systems available to identify the various domains or regions of a TCR.

In certain embodiments, the TCR may be a heterodimer of two chains α and β (or optionally γ and δ) that are linked, such as by a disulfide bond or disulfide bonds. In certain embodiments, the constant domain of the TCR may contain short connecting sequences in which a cysteine residue forms a disulfide bond, thereby linking the two chains of the TCR. In certain embodiments, a TCR may have an additional cysteine residue in each of the α and β chains, such that the TCR contains two disulfide bonds in the constant domains. In certain embodiments, each of the constant and variable domains contain disulfide bonds formed by cysteine residues.

In certain embodiments, the TCR is one generated from a known TCR sequence(s), such as sequences of Vα,β chains, for which a substantially full-length coding sequence is readily available. Methods for obtaining full-length TCR sequences, including V chain sequences, from cell sources are well known. In certain embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In certain embodiments, the TCR is obtained from a biological source, such as from cells such as from a T cell (e.g. cytotoxic T cell), T cell hybridomas or other publicly available source. In certain embodiments, the T cells can be obtained from in vivo isolated cells. In certain embodiments, the T cells can be obtained from a cultured T cell hybridoma or clone. In certain embodiments, the TCR or antigen-binding portion thereof can be synthetically generated from knowledge of the sequence of the TCR. In certain embodiments, a high-affinity T cell clone for a target antigen (e.g., a cancer antigen) is identified, isolated from a patient, and introduced into the cells. In certain embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808). In certain embodiments, phage display is used to isolate TCRs against a target antigen (see, e.g., Varela-Rohena et al. (2008) Nat Med. 14: 1390-1395 and Li (2005) Nat Biotechnol. 23:349-354).

In certain embodiments, the TCR or antigen-binding portion thereof is one that has been modified or engineered. In certain embodiments, directed evolution methods are used to generate TCRs with altered properties, such as with higher affinity for a specific MHC-peptide complex. In certain embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In certain embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In certain embodiments, the TCR can contain an introduced disulfide bond or bonds. In certain embodiments, the native disulfide bonds are not present. In certain embodiments, the one or more of the native cysteines (e.g. in the constant domain of the α chain and β chain) that form a native interchain disulfide bond are substituted with another residue, such as with a serine or alanine. In certain embodiments, an introduced disulfide bond can be formed by mutating non-cysteine residues on the alpha and beta chains, such as in the constant domain of the α chain and β chain, to cysteine. Exemplary non-native disulfide bonds of a TCR are described in PCT Publication Nos. WO2006/000830 and WO2006/037960, the disclosures of which are incorporated herein by reference in their entirety. In certain embodiments, cysteines can be introduced at residue Thr48 of the α chain and Ser57 of the β chain, at residue Thr45 of the α chain and Ser77 of the β chain, at residue Tyr10 of the α chain and Ser17 of the β chain, at residue Thr45 of the α chain and Asp59 of the β chain and/or at residue Ser15 of the α chain and Glu15 of the β chain. In certain embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In certain embodiments, the TCR chains contain a transmembrane domain. In some embodiments, the transmembrane domain is positively charged. In certain embodiments, the TCR chain contains a cytoplasmic tail. In certain embodiments, each chain (e.g. alpha or beta) of the TCR can possess one N-terminal immunoglobulin variable domain, one immunoglobulin constant domain, a transmembrane region, and a short cytoplasmic tail at the C-terminal end. In certain embodiments, a TCR, for example via the cytoplasmic tail, is associated with invariant proteins of the CD3 complex involved in mediating signal transduction. In certain embodiments, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3y, CD35, CD3s and CD3ζ chains) contain one or more immunoreceptor tyrosine-based activation motif or ITAM that are involved in the signaling capacity of the TCR complex.

In certain embodiments, the TCR is a full-length TCR. In certain embodiments, the TCR is an antigen-binding portion. In certain embodiments, the TCR is a dimeric TCR (dTCR). In certain embodiments, the TCR is a single-chain TCR (sc-TCR). A TCR may be cell-bound or in soluble form. In certain embodiments, the TCR is in cell-bound form expressed on the surface of a cell. In certain embodiments, a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In certain embodiments, the bond can correspond to the native interchain disulfide bond present in native dimeric αβ TCRs. In certain embodiments, the interchain disulfide bonds are not present in a native TCR. For example, in certain embodiments, one or more cysteines can be incorporated into the constant region extracellular sequences of dTCR polypeptide pair. In certain embodiments, both a native and a non-native disulfide bond may be desirable. In certain embodiments, the TCR contains a transmembrane sequence to anchor to the membrane. In certain embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In certain embodiments, the TCR is an scTCR, which is a single amino acid strand containing an α chain and a β chain that is able to bind to MHC-peptide complexes. Typically, an scTCR can be generated using methods known to those of skill in the art, see, e.g., PCT Publication Nos. WO 96/13593, WO 96/18105, WO 99/18129, WO 04/033685, WO 2006/037960, WO 2011/044186; U.S. Pat. No. 7,569,664; and Schlueter, C. J. et al. J. Mol. Biol. 256, 859 (1996). In certain embodiments, an scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment. In certain embodiments, an scTCR contains a first segment constituted by an amino acid sequence corresponding to a TCR β chain variable region, a second segment constituted by an amino acid sequence corresponding to a TCR α chain variable region sequence fused to the N terminus of an amino acid sequence corresponding to a TCR α chain constant domain extracellular sequence, and a linker sequence linking the C terminus of the first segment to the N terminus of the second segment. In certain embodiments, an scTCR contains a first segment constituted by an a chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment. In certain embodiments, an scTCR contains a first segment constituted by a TCR β chain variable region sequence fused to the N terminus of a β chain extracellular constant domain sequence, and a second segment constituted by an a chain variable region sequence fused to the N terminus of a sequence comprising an α chain extracellular constant domain sequence and transmembrane sequence, and, optionally, a linker sequence linking the C terminus of the first segment to the N terminus of the second segment. In certain embodiments, for the scTCR to bind an MHC-peptide complex, the α and β chains must be paired so that the variable region sequences thereof are orientated for such binding. Various methods of promoting pairing of an α and β in an scTCR are well known in the art. In certain embodiments, a linker sequence is included that links the α and β chains to form the single polypeptide strand. In certain embodiments, the linker should have sufficient length to span the distance between the C terminus of the α chain and the N terminus of the β chain, or vice versa, while also ensuring that the linker length is not so long so that it blocks or reduces bonding of the scTCR to the target peptide-MHC complex. In certain embodiments, the linker of an scTCR that links the first and second TCR segments can be any linker capable of forming a single polypeptide strand, while retaining TCR binding specificity. In certain embodiments, the linker sequence may, for example, have the formula -P-AA-P-, wherein P is proline and AA represents an amino acid sequence wherein the amino acids are glycine and serine. In certain embodiments, the first and second segments are paired so that the variable region sequences thereof are orientated for such binding. In certain embodiments, the linker can contain from or from about 10 to 45 amino acids, such as 10 to 30 amino acids or 26 to 41 amino acids residues, for example 29, 30, 31 or 32 amino acids. In certain embodiments, an scTCR contains a disulfide bond between residues of the single amino acid strand, which, in some cases, can promote stability of the pairing between the α and β regions of the single chain molecule (see e.g. U.S. Pat. No. 7,569,664). In certain embodiments, the scTCR contains a covalent disulfide bond linking a residue of the immunoglobulin region of the constant domain of the α chain to a residue of the immunoglobulin region of the constant domain of the β chain of the single chain molecule. In certain embodiments, the disulfide bond corresponds to the native disulfide bond present in a native dTCR. In certain embodiments, the disulfide bond in a native TCR is not present. In certain embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described above. In some cases, both a native and a non-native disulfide bond may be present.

In certain embodiments, any of the TCRs, including a dTCR or an scTCR, can be linked to signaling domains that yield an active TCR on the surface of a T cell. In certain embodiments, the TCR is expressed on the surface of cells. In certain embodiments, the TCR contains a sequence corresponding to a transmembrane sequence. In certain embodiments, the transmembrane domain can be a Ca or CP transmembrane domain. In certain embodiments, the transmembrane domain can be from a non-TCR origin, for example, a transmembrane region from CD3z, CD28 or B7.1. In certain embodiments, the TCR contains a sequence corresponding to cytoplasmic sequences. In certain embodiments, the TCR contains a CD3z signaling domain. In certain embodiments, the TCR is capable of forming a TCR complex with CD3. In certain embodiments, the TCR or antigen binding portion thereof may be a recombinantly produced natural protein or mutated form thereof in which one or more property, such as binding characteristic, has been altered. In certain embodiments, a TCR may be derived from one of various animal species, such as human, mouse, rat, or other mammals.

In certain embodiments, the TCR has affinity to a target antigen on a target cell. The target antigen may include any type of protein, or epitope thereof, associated with the target cell. For example, the TCR may comprise affinity to a target antigen on a target cell that indicates a particular disease state of the target cell. In certain embodiments, the target antigen is processed and presented by MHCs.

In certain embodiments, the immune receptor (e.g., TCR) provides specificity to the immune cell towards a target antigen. In certain embodiments, the TCR (e.g., exogenous TCR) provided target antigen specificity is the same as the target antigen that the immune cell is specific for. In such embodiments, the TCR specificity is said to be matched with the endogenous specificity of the immune cell. In certain embodiments, the TCR (e.g., exogenous TCR) provided target antigen specificity is different to the target antigen that the immune cell is specific for. In such embodiments, the TCR specificity is said to be unmatched with the endogenous specificity of the immune cell. As such, a TCR having unmatched specificity with the endogenous specificity of the immune cell gives rise to a multispecific (e.g., bispecific) immune cell.

In certain embodiments, the NK-TCR cell is generated by genetically modifying an allogeneic NK cell from one of more donors with a recombinant TCR to form an allogeneic NK-TCR cells. In certain embodiments, the allogeneic NK-TCR cells is stimulated with a cell of leukemic origin that has been loaded with exogenous MHC class I antigen or an MHC class II antigen using the methods disclosed above.

### F. Stimulation and Expansion of NK Cells

In certain exemplary embodiments, the foregoing conditions for stimulating and expanding NK cells, may be provided by a modified cell of leukemic origin as described herein. Accordingly, provided herein is a method for activating and expanding a population of immune cells (e.g., NK cells), comprising: obtaining a population of immune cells comprising NK cells; contacting the population of NK cells with a modified cell of leukemic origin; and culturing the population of NK cells under conditions suitable to stimulate proliferation of the NK immune cells, thereby activating and expanding the population of NK cells.

Due to the nature of the modified cell of leukemic origin, methods utilizing the modified cell of leukemic origin result in the enhanced generation of certain subsets of immune cells. In certain embodiments, provided herein are methods for generating a population of memory immune cells (e.g., memory NK cells). Accordingly, provided herein is a method for generating a population of memory immune cells (e.g., memory NK cells), comprising: obtaining a population of cells comprising immune cells (e.g., NK cells); contacting the population of immune cells with a modified cell of leukemic origin; and culturing the population of cells under conditions suitable to stimulate proliferation of the immune cells, thereby generating the population of memory immune cells. In certain embodiments, provided herein are methods for generating a population of memory immune cells (e.g., memory NK cells). Accordingly, provided herein is a method for generating a population of memory NK cells, comprising:

obtaining a population of cells comprising immune cells (e.g., NK cells); contacting the population of cells with a modified cell of leukemic origin; and culturing the population of cells under conditions suitable to stimulate proliferation of the immune cells, thereby generating the population of memory NK cells. As such, the methods provided herein can be used to enrich the memory NK cell population from a source, e.g., peripheral blood. Memory NK cells are long-lived and can quickly expand to generate a large number of effector NK cells upon exposure to activating ligands. In general, memory NK cells are characterized by the presence of certain cell surface markers, including, NKG2C and CD57. Precursor memory NK cells are characterized by the presence of NKG2C and lack of CD57.

In the various methods provided herein for stimulating and expanding immune cells such as NK cells, conditions suitable to stimulate proliferation of the immune cells comprises providing a modified cell of leukemic origin that exhibits a mature dendritic cell phenotype. In certain exemplary embodiments, the modified cell of leukemic origin is non-proliferating (e.g., via irradiation).

Cytokines such as IL-2 and/or IL-15 may be added to the co-culture medium to enhance the expansion and activation of the NK cells by the modified cell of leukemic origin. In addition, the immune cells (e.g., NK cells) are maintained under conditions necessary to support growth, for example, an appropriate temperature (e.g., 37° C.) and atmosphere (e.g., air plus 5% CO 2). Immune cells (e.g., NK cells) that have been exposed to varied stimulation times may exhibit different characteristics.

The population of immune cells (e.g., NK cells, memory NK cells) generated by the methods disclosed herein can be multiplied by about 10 fold, 20 fold, 30 fold, 40 fold, 50 fold, fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the NK cells expand in the range of about 20 fold to about 80 fold.

Following culturing, the immune cells (e.g., NK cells) can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In certain exemplary embodiments, the level of confluence is 90% or greater. A period of time can be any time suitable for the culture of cells in vitro. The cell medium may be replaced during the culture of the immune cells at any time. In certain exemplary embodiments, the cell medium is replaced about every 2 to 3 days.

The immune cells are then harvested from the culture apparatus whereupon the immune cells can be used immediately or cryopreserved to be stored for use at a later time. In certain embodiments, methods provided herein further include cryopreserving the resulting immune cell population. In embodiments where the stimulated and expanded immune cells are for use in downstream modification, fresh or cryopreserved immune cells are prepared for the introduction of genetic material into the immune cells (e.g., nucleic acids encoding an immune receptor, e.g., TCR or CAR). In certain embodiments, cryopreserved immune cells are thawed prior to the introduction of genetic material. In certain embodiments, fresh or cryopreserved immune cells are prepared for electroporation with RNA encoding an immune receptor (e.g., TCR or CAR).

Another procedure for ex vivo expansion of immune cells is described in U.S. Pat. No. 5,199,942, the disclosure of which is incorporated by reference herein in its entirety. Methods for expanding and activating immune cells can also be found in U.S. Pat. Nos. 7,754,482, 8,722,400, and 9,555,105, the disclosures of which are incorporated herein in their entirety. Such art recognized expansion and activation methods can be an alternative or in addition to the methods described herein.

The culturing step (e.g., contact with a modified cell of leukemic origin as described herein) can be short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step (e.g., contact with a modified cell of leukemic origin as described herein) can be longer, for example 1, 2, 3, 4, 5, 6, 7, 8, 9, 10, 11, 12, 13, 14, or more days.

In certain embodiments, the cells may be cultured for several hours (about 3 hours) to about 14 days or any hourly integer value in between. Conditions appropriate for immune cell (e.g., T cell) culture include an appropriate media (e.g., Minimal Essential Media or RPMI Media 1640 or, X-vivo 15, (Lonza)) that may contain factors necessary for proliferation and viability, including serum (e.g., fetal bovine or human serum), insulin, IFNγ, interleukin-2 (IL-2), IL-4, IL-7, IL-10, IL-15, GM-CSF, TGF8, and TNF-α, or any other additives for the growth of cells known to the skilled artisan. For example, other additives may include, without limitation, surfactant, plasmanate, and reducing agents such as N-acetyl-cysteine and 2-mercaptoethanol. Media can include RPMI 1640, AIM-V, DMEM, MEM, a-MEM, F-12, X-Vivo 10, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject.

### G. Modified NK Cells and Methods of Producing the Same

Also provided are methods for producing or generating a modified population of expanded and activated NK cells. In particular, following expansion and activation of NK cells according to the methods of the invention, expanded and activated NK cells may be further modified by introducing one or more nucleic acids encoding an exogenous immune receptor (e.g., a TCR or CAR).

In certain embodiments, the immune receptor (e.g., TCR and/or CAR) is introduced into the NK cell by an expression vector. Expression vectors comprising a nucleic acid sequence encoding a TCR and/or CAR are known in the art. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, piggyBac, and Integrases such as Phi31. Some other suitable expression vectors include Herpes simplex virus (HSV) and retrovirus expression vectors.

In certain embodiments, the nucleic acid encoding an immune receptor is introduced into the NK cell via viral transduction. In certain embodiments, the viral transduction comprises contacting the immune or precursor cell with a viral vector comprising the nucleic acid encoding the immune receptor.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the immune receptor in the host cell. In certain embodiments, the adenovirus genome is a 36 kb, linear, double stranded DNA, where a foreign DNA sequence (e.g., a nucleic acid encoding an exogenous TCR and/or CAR) may be inserted to substitute large pieces of adenoviral DNA in order to make the expression vector of the present disclosure (see, e.g., Danthinne and Imperiale, Gene Therapy (2000) 7(20): 1707-1714).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid (e.g., a nucleic acid encoding an exogenous TCR and/or CAR) into the viral genome at certain locations to produce a virus that is replication defective. Though the retroviral vectors are able to infect a broad variety of cell types, integration and stable expression of the TCR and/or CAR requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the human immunodeficiency viruses (e.g., HIV-1, HIV-2) and the simian immunodeficiency virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression, e.g., of a nucleic acid encoding a TCR and/or CAR (see, e.g., U.S. Pat. No. 5,994,136).

Expression vectors can be introduced into a NK cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The genetically modified NK cells may then be expanded and screened by virtue of a marker present in the vectors. Various markers that may be used are known in the art, and may include hprt, neomycin resistance, thymidine kinase, hygromycin resistance, etc.

Modified NK cells may be produced by stably transfecting host cells with an expression vector including a nucleic acid of the present disclosure. Additional methods for generating a modified NK cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing an immune receptor may be expanded ex vivo.

Physical methods for introducing an expression vector into NK cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

In some embodiments, a nucleic acid encoding an immune receptor is RNA, e.g., in vitro synthesized RNA. Methods for in vitro synthesis of RNA are known in the art; any known method can be used to synthesize RNA comprising a sequence encoding an immune receptor (e.g., TCR and/or CAR). Methods for introducing RNA into a host cell are known in the art. See, e.g., Zhao et al. Cancer Res. (2010) 15: 9053. Introducing RNA comprising a nucleotide sequence encoding a TCR and/or CAR into a host cell can be carried out in vitro, ex vivo or in vivo. For example, an NK cell can be electroporated in vitro or ex vivo with RNA comprising a nucleotide sequence encoding a CAR.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In certain embodiments, the NK cells can be incubated or cultivated prior to, during and/or subsequent to introducing the nucleic acid molecule encoding the immune receptor (e.g., CAR). The cells (e.g., NK cells) can be incubated or cultivated prior to, during or subsequent to the introduction of the nucleic acid molecule encoding the immune receptor, such as prior to, during or subsequent to the transduction of the cells with a viral vector (e.g. lentiviral vector) encoding the immune receptor. In certain embodiments, the method includes activating or stimulating cells with a stimulating or activating agent (e.g. a modified cell of leukemic origin) prior to introducing the nucleic acid molecule encoding the immune receptor. In certain embodiments, the method includes activating or stimulating cells with a stimulating or activating agent (e.g. a modified cell of leukemic origin) after introducing the nucleic acid molecule encoding the immune receptor.

### H. Methods of Treatment

In certain embodiments, immune cells obtained according to the methods of the disclosure may be subsequently employed in an adoptive cell therapy. Adoptive cell therapy is an immunotherapy in which immune cells (e.g., NK cells) are given to a subject to fight diseases, such as cancer. In general, T cells can be obtained from the subject's own peripheral blood or tumor tissue, stimulated and expanded ex vivo according to the methods of the disclosure, and then administered back to the subject (i.e., autologous adoptive cell therapy). In other embodiments, NK cells can be obtained from a first subject (e.g., from peripheral blood or tumor tissue of the first subject), stimulated and expanded ex vivo according to the methods of the disclosure, and then administered to a second subject (i.e., allogeneic adoptive cell therapy).

In certain embodiments, the NK cells can be further modified ex vivo (e.g., genetically modified) to express an immune receptor (e.g., a CAR). The term “adoptive cell therapy” refers to both NK cell therapy without genetic modification, and NK cell therapy with genetic modification to, e.g., express an immune receptor.

As such, in certain embodiments, provided herein is a method for treating a disease or disorder in a subject in need thereof, comprising administering a composition comprising a modified immune cell of the disclosure, wherein the modified immune cell comprises an immune receptor. In certain embodiments, the immune receptor is a CAR as described elsewhere herein.

In certain embodiments, the disease or disorder is a cancer. In certain embodiments, the cancer is a tumor. In certain embodiments, the cancer is a liquid tumor, or a solid tumor. In certain embodiments, the disease or disorder is an infectious disease.

In other aspects, provided herein is a method for treating a tumor in a subject in need thereof, comprising administering to the subject a modified NK cell produced by any one of the methods described herein.

The methods of the invention results in an NK with improved properties for cell therapy. In certain embodiments, co-culturing NK cells with a modified cell of leukemic origin increases the viability and activation of NK cells for use in vivo. In certain embodiments, co-culturing the NK cell with the cell of leukemic origin activates the immune cell.

In other embodiments, co-culturing NK cells with an antigen-loaded modified cell of leukemic origin enriches for antigen-specific immune cells. In certain embodiments, the antigen-loaded modified cell of leukemic origin redirects the specificity of the NK cell to the antigen. In certain embodiments, redirection of the specificity of the NK cell is accomplished by inducing the production of or enriching NK cells having exogenous TCRs directed to the antigen. As such, in certain embodiments, co-culturing an antigen-loaded modified cell of leukemic origin with a modified NK cells expressing a TCR results in NK cells comprising expressing TCR having specificity for the antigen.

In certain embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In certain embodiments, the first and second subjects are genetically identical. In certain embodiments, the first and second subjects are genetically similar. In certain embodiments, the second subject expresses the same HLA class or supertype as the first subject.

In certain embodiments, the subject has been treated with a therapeutic agent targeting the disease or condition, e.g. the tumor, prior to administration of the cells or composition containing the cells. In certain embodiments, the subject is refractory or non-responsive to the other therapeutic agent. In certain embodiments, the subject has persistent or relapsed disease, e.g., following treatment with another therapeutic intervention, including chemotherapy, radiation, and/or hematopoietic stem cell transplantation (HSCT), e.g., allogenic HSCT. In certain embodiments, the administration effectively treats the subject despite the subject having become resistant to another therapy.

In certain embodiments, the subject is responsive to the other therapeutic agent, and treatment with the therapeutic agent reduces disease burden. In certain embodiments, the subject is initially responsive to the therapeutic agent, but exhibits a relapse of the disease or condition over time. In certain embodiments, the subject has not relapsed. In such embodiments, the subject is determined to be at risk for relapse, such as at a high risk of relapse, and thus the cells are administered prophylactically, e.g., to reduce the likelihood of or prevent relapse. In certain embodiments, the subject has not received prior treatment with another therapeutic agent.

In certain embodiments, the subject has persistent or relapsed disease, e.g., following treatment with another therapeutic intervention, including chemotherapy, radiation, and/or hematopoietic stem cell transplantation (HSCT), e.g., allogenic HSCT. In certain embodiments, the administration effectively treats the subject despite the subject having become resistant to another therapy.

The NK cells generated by the methods of the invention can administered to an animal, e.g., a mammal, e.g., a human, to treat a disease or disorder, e.g., a cancer. In addition, the cells of the present disclosure can be used for the treatment of any condition related to a cancer, especially a cell-mediated immune response against a tumor cell(s), where it is desirable to treat or alleviate the disease. The types of cancers to be treated using a method disclosed herein may be non-solid tumors (such as hematological tumors) or solid tumors. Adult tumors/cancers and pediatric tumors/cancers are also included. In certain embodiments, the cancer is a solid tumor or a hematological tumor. In certain embodiments, the cancer is a carcinoma. In certain embodiments, the cancer is a sarcoma. In certain embodiments, the cancer is a leukemia. In certain embodiments, the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas).

In certain embodiments, the NK cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as memory NK cells, for immune cell administration. In certain embodiments, the dosage of cells is based on a desired total number (or number per kg of body weight) of cells in the individual populations or of individual cell types. In certain embodiments, the dosage is based on a combination of such features, such as a desired number of total cells, desired ratio, and desired total number of cells in the individual populations.

In certain embodiments, for the administration of NK cells, the populations or sub-types of cells, such as memory NK cells, are administered at or within a tolerated difference of a desired dose of total cells, such as a desired dose of NK cells.

In certain embodiments, the desired dose is a desired number of cells or a desired number of cells per unit of body weight of the subject to whom the cells are administered, e.g., cells/kg. In certain embodiments, the desired dose is at or above a minimum number of cells or minimum number of cells per unit of body weight. In certain embodiments, among the total cells, administered at the desired dose, the individual populations or sub-types are present at or near a desired output ratio, e.g., within a certain tolerated difference or error of such a ratio.

In certain embodiments, the cells are administered at or within a tolerated difference of a desired dose of one or more of the individual populations or sub-types of cells, such as a desired dose of memory NK cells. In certain embodiments, the desired dose is a desired number of cells of the sub-type or population, or a desired number of such cells per unit of body weight of the subject to whom the cells are administered, e.g., cells/kg. In certain embodiments, the desired dose is at or above a minimum number of cells of the population or subtype, or minimum number of cells of the population or sub-type per unit of body weight. Thus, in certain embodiments, the dosage is based on a desired fixed dose of total cells and a desired ratio, and/or based on a desired fixed dose of one or more, e.g., each, of the individual sub-types or sub-populations. Thus, in certain embodiments, the dosage is based on a desired fixed or minimum dose of NK cells and a desired ratio of memory and precursor NK cells, and/or is based on a desired fixed or minimum dose of memory NK cells.

In certain embodiments, the cells (e.g., modified cells of leukemic origin, and/or immune cells comprising an immune receptor), or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about billion cells, about 40 billion cells, about 50 million cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In certain embodiments, the dose of total cells (e.g., modified cells of leukemic origin, and/or immune cells comprising an immune receptor) and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in certain embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In certain embodiments, a suitable dosage range of cells for use in a method provided herein includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg.

In certain embodiments, the cells (e.g., immune cells comprising an immune receptor) are administered at or within a certain range of error of between at or about 10⁴ and at or about 10⁹ NK cells/kilograms (kg) body weight, such as between 10⁵ and 10⁶ CD4⁺ and/or CD8⁺ cells/kg body weight, for example, at or about 1×10⁵ NK cells/kg, 1.5×10⁵ NK cells/kg, 2×10⁵ NK cells/kg, or 1×10⁶ NK cells/kg body weight.

In certain embodiments, for the administration of NK cells (e.g., NK CARs), the cells are administered at or within a tolerated range of a desired output ratio of multiple cell populations or sub-types. In certain embodiments, the desired ratio can be a specific ratio or can be a range of ratios, for example, in some embodiments, the desired ratio (e.g., ratio of memory and precursor NK cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4: 1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In certain embodiments, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In certain embodiments, a dose of NK cells is administered to a subject in need thereof, in a single dose or multiple doses. In certain embodiments, a dose of cells is administered in multiple doses, e.g., once a week or every 7 days, once every 2 weeks or every 14 days, once every 3 weeks or every 21 days, once every 4 weeks or every 28 days.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In certain embodiments, the NK cells are administered as part of a combination treatment, such as simultaneously with or sequentially with, in any order, another therapeutic intervention, such as an antibody or engineered cell or receptor or agent, such as a cytotoxic or therapeutic agent. The NK cells in certain embodiments are co-administered with one or more additional therapeutic agents or in connection with another therapeutic intervention, either simultaneously or sequentially in any order. In certain embodiments, the cells are co-administered with another therapy sufficiently close in time such that the cell populations enhance the effect of one or more additional therapeutic agents, or vice versa. In certain embodiments, the cells are administered prior to the one or more additional therapeutic agents. In certain embodiments, the cells are administered after the one or more additional therapeutic agents. In certain embodiments, the one or more additional agents includes a cytokine, such as IL-2 or IL-15, for example, to enhance persistence. In certain embodiments, the methods comprise administration of a chemotherapeutic agent.

Following administration of the NK cells, the biological activity of the engineered cell populations in some embodiments is measured, e.g., by any of a number of known methods. Parameters to assess include specific binding of a modified or natural NK cell or other immune cell to antigen, in vivo, e.g., by imaging, or ex vivo, e.g., by ELISA or flow cytometry. In certain embodiments, the ability of the modified immune cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In certain embodiments the biological activity is measured by assessing clinical outcome, such as reduction in tumor burden or load, or reduction in the occurrence of relapse.

In certain embodiments, the subject is provided a secondary treatment. Secondary treatments include but are not limited to chemotherapy, radiation, surgery, and medications.

In certain embodiments, the subject can be administered conditioning therapy prior to adoptive cell therapy. In certain embodiments, the conditioning therapy comprises administering an effective amount of cyclophosphamide to the subject. In certain embodiments, the conditioning therapy comprises administering an effective amount of fludarabine to the subject. In certain embodiments, the conditioning therapy comprises administering an effective amount of a combination of cyclophosphamide and fludarabine to the subject. Administration of a conditioning therapy prior to adoptive cell therapy may increase the efficacy of the adoptive cell therapy. Methods of conditioning patients for adoptive cell therapy are described in U.S. Pat. No. 9,855,298, which is incorporated herein by reference in its entirety.

NK cells of the disclosure can be administered in dosages and routes and at times to be determined in appropriate pre-clinical and clinical experimentation and trials. Cell compositions may be administered multiple times at dosages within these ranges. Administration of the cells of the disclosure may be combined with other methods useful to treat the desired disease or condition as determined by those of skill in the art.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use the methods and compositions featured in the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The discovery of a subset of long-lived adaptive/memory NKG2C/CD57-expressing NK cells in CMV-seropositive individuals has revolutionized the field of NK cell biology. Several features render adaptive NK cells a potentially attractive contributor to the efficacy of tumor-targeting monoclonal antibodies (Capuano C, et al. Front Immunol. 2018; 9: 1031) and predict a lower sensitivity to immunosuppressive signals in the tumor microenvironment (Sarhan D, et al. Cancer Res. 2016; 76(19):5696-5706; Sarhan D, et al. Cancer Immunol Res. 2018; 6(7):766-775). The requirements for adaptive NK cell expansion ex vivo have however not been fully characterized. Ex vivo expansion of adaptive NK cells can be achieved by coculturing NK cells from CMV-positive subjects with CMV-infected fibroblasts+IL-2 (Gums M, et al. Blood. 2006; 107(9):3624-31). HLA-E-transfected tumor cell lines+IL-15 (Liu L, et al. Cancer Immunol Res. 2017; 5(8):654-665) or IgG-opsonized tumor cells+IL-2 (Capuano C, et al. Front Immunol. 2018; 9: 1031). However, the reported fold-expansion after 10-14 days in culture is generally below 30-fold.

Studies were performed to investigate ex vivo expansion of adaptive NK cells using mature dendritic cells (DCOne mDCs) derived from the DCOne leukemic cell line.

### Example 1: DCOne mDCs Maintain High Cell Viability, Increase NK Cell Frequency and Induce Production of Immune-Cell-Recruiting Chemokines, Proinflammatory and Effector Cytokines when Co-Cultured with NK Cells

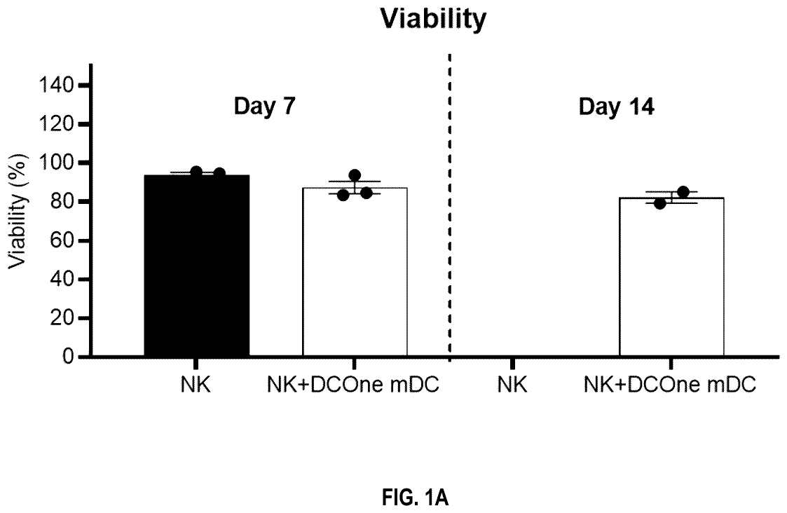

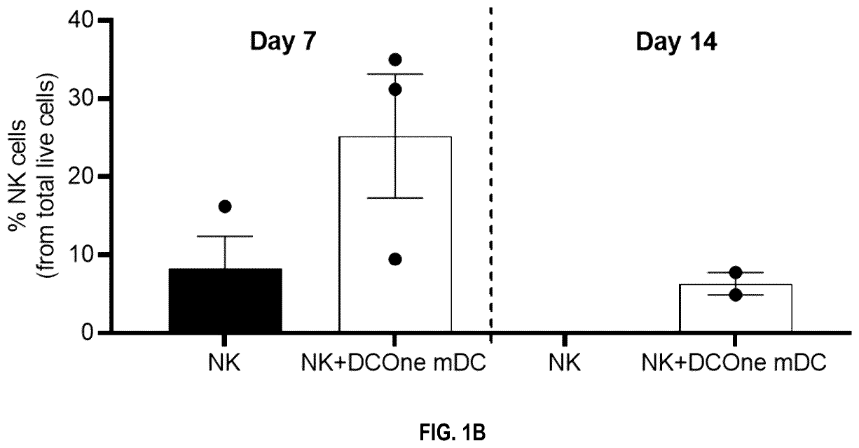

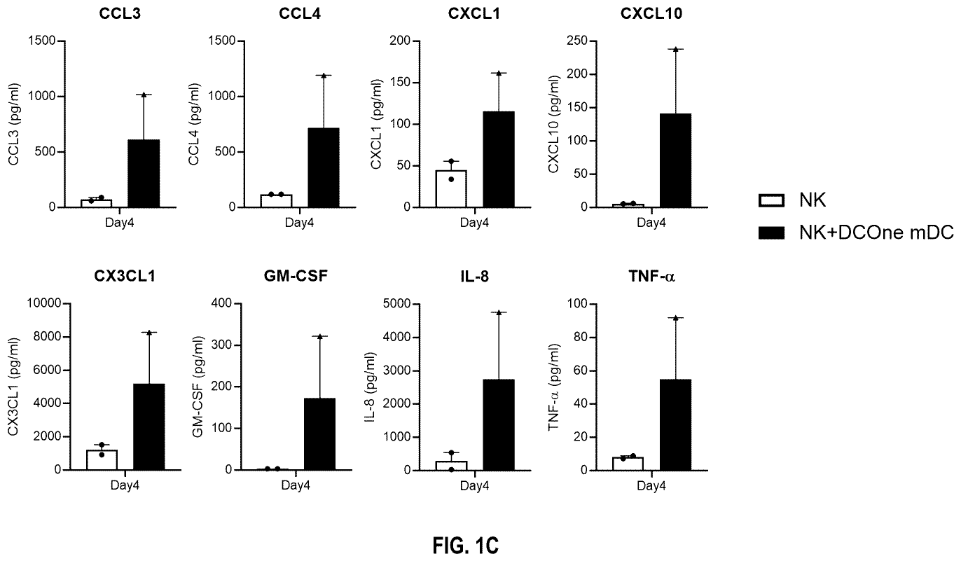

FIG. 1A shows high viability of cells when co-cultured with DCOne mDC for 7 or 14 days without addition of cytokines. Cells were harvested and assessed for viability using Nucleo Counter NC-200. The viability of NK cells was enhanced when co-cultured with DCOne mDC for 7 or 14 days without addition of cytokines with the more prominent effect observed at Day 14.

FIG. 1B shows the frequency of NK cells on day 7 and 14 co-cultures of NK cells and DCOne mDC. At day 7 and 14, cells were harvested and stained with anti-CD56, CD3, NKG2C and CD57 specific antibodies and analyzed by flow cytometry. The percentage of NK cells relative to total live cells is shown. As shown, the frequency of NK cells was significantly increased by the presence of DCOne mDC.

FIG. 1C shows induced production of immune-cell-recruiting chemokines, proinflammatory and effector cytokines when NK cells are co-cultured with DCOne-derived cells. Supernatants from NK cells co-cultured in the presence or absence DCOne mDCs were harvested on day 4. Multi-analyte profiling of cytokines and chemokines was performed using the Luminex MAGPIX® system (Luminex Corporation, USA). The levels of cytokines and chemokines were determined using magnetic antibody-coated beads (R&D). All analyses were performed according to the manufacturers' protocols. Acquired fluorescence data were analyzed by the 4.3×PONENT software (Luminex). As shown in FIG. 1C, production of all tested immune-cell-recruiting chemokines, proinflammatory and effector cytokines was induced.

### Example 2: In Vitro Expansion of Memory (Adaptive) NK Cells by DCOne mDC in the Presence of Cytokines

Study was performed to investigate in vitro expansion of memory NK cells by co-culturing the memory NK cells with or without DCOne mDC in the presence or absence of IL-2, IL-15, combination of IL-2 and IL-15, combination of IL-2 and IL-21, or combination of IL-15 and IL-21 for 2 weeks. The NK cells were enriched from PBMCs isolated from buffy coats of CMV-positive healthy donors.

### Example 2-1: Increased NK Cell Frequency and Expansion in DCOne mDC-NK Cell 14-Day Co-Cultures

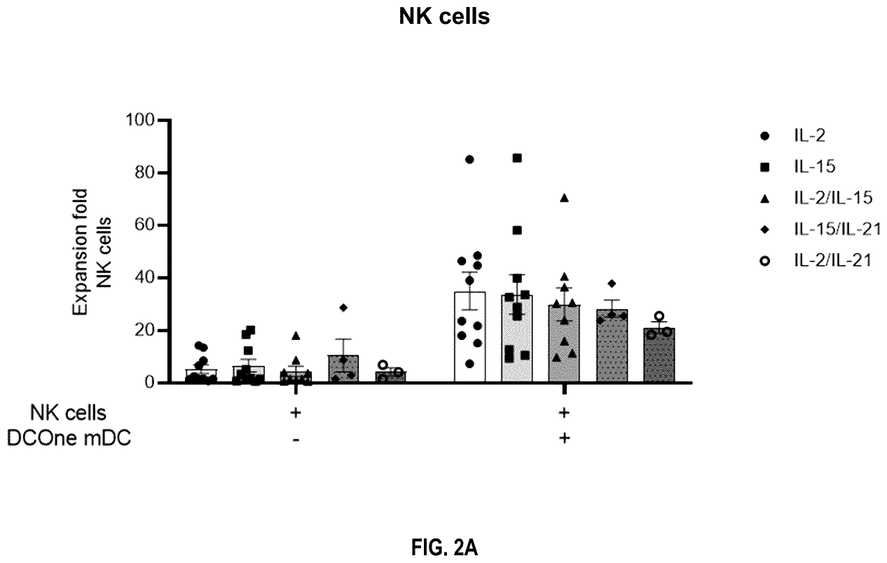

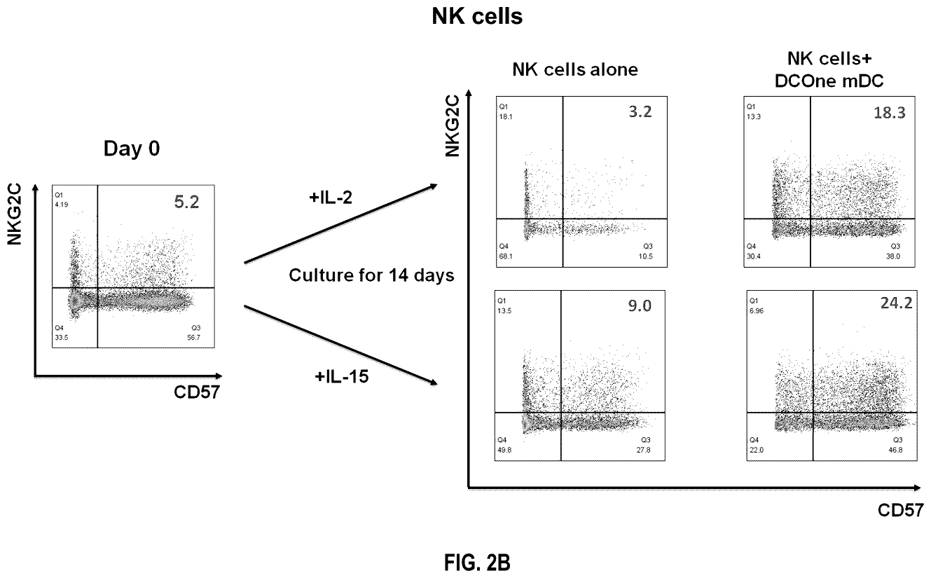

FIG. 2A shows the expansion of NK cells on day 14 co-cultures of NK cells and DCOne mDC, compared to the cultures of NK cells alone without DCOne mDC, in the absence and/or presence of cytokines IL-2, IL-15, a combination of IL-2 and IL-15, IL-2 and IL-21, or IL-15 and IL-21. At day 14 cells were harvested and stained with anti-CD56, CD3, NKG2C and CD57 specific antibodies and analyzed by flow cytometry. As shown in FIG. 2A, DCOne mDC and cytokines IL-2, IL-15, the combination of IL-1 and 11-15, the combination of IL-2 and IL-21, and the combination of 11-5 and IL-21 significantly increased NK cell frequency and expansion, by as much as 60-80 folds.

FIG. 2B is a set of images illustrating the flow cytometry analyses of NKG2C+/CD57+ NK cells at day 0 in CD56+/CD3− NK cells from one representative donor followed by 14 days expansion with IL-2 or IL-15, with or without DCOne derived mDCs, according to an experimental example. At day 14 of co-culturing, cells were harvested and stained with anti-CD56, CD3, NKG2C and CD57 specific antibodies and analyzed by flow cytometry. As shown in FIG. 2B, co-culturing with DCOne mDC significantly improved the expansion of NKG2C+/CD57+NK cells in the presence of cytokine IL-2 or IL-15.

### Example 2-2: Co-Culture of NK Cells with DCOne mDC Induced Increased Expansion of Memory (NKG2C+/CD57+) NK Cells

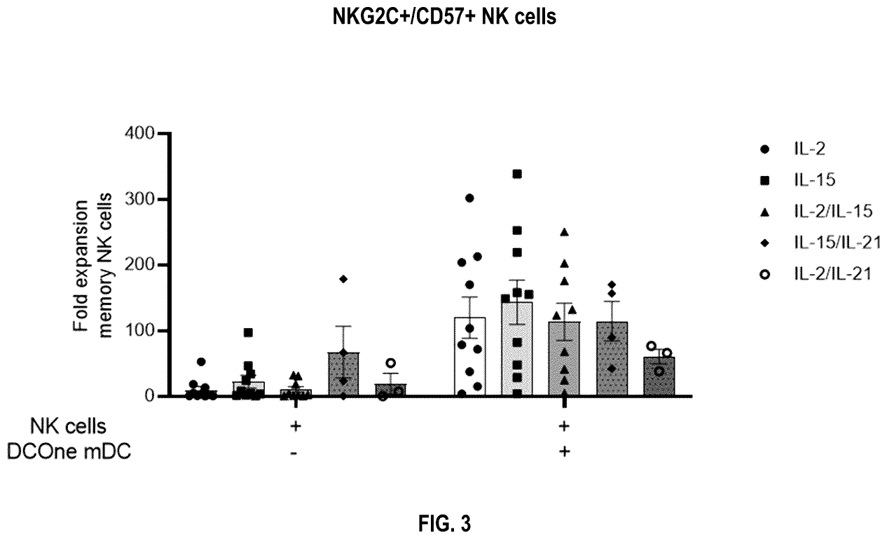

In order to determine the specific effects of DCOne mDC on memory (NKG2C+/CD57+) NK cells, memory NK specific staining was used to analyze the expansion of this sub-population. FIG. 3 shows the expansion of memory (NKG2C+/CD57+) NK cells on day 14 co-cultures of NK cells and DCOne mDC, compared to memory (NKG2C+/CD57+) NK cells cultured without DCOne mDC, in the absence and/or presence of cytokines IL-2, IL-15, or a combination of IL-2 and IL-15, IL-2 and IL-21, or IL-15 and IL-21. At day 14 cells were harvested and stained with anti-CD56, CD3, NKG2C and CD57 specific antibodies and analyzed by flow cytometry. As shown in FIG. 3, co-culturing the NK cells with DCOne mDC significantly increases the NK cell expansion induced by cytokines such as IL-2, IL-15, or the combinations of IL-1 and 11-15, IL-2 and IL-21, and 11-5 and IL-21.

### Example 2-3: Increased Precursor Memory NK Cell Frequency and Expansion in DCOne mDC-NK Cell 14-Day Co-Cultures

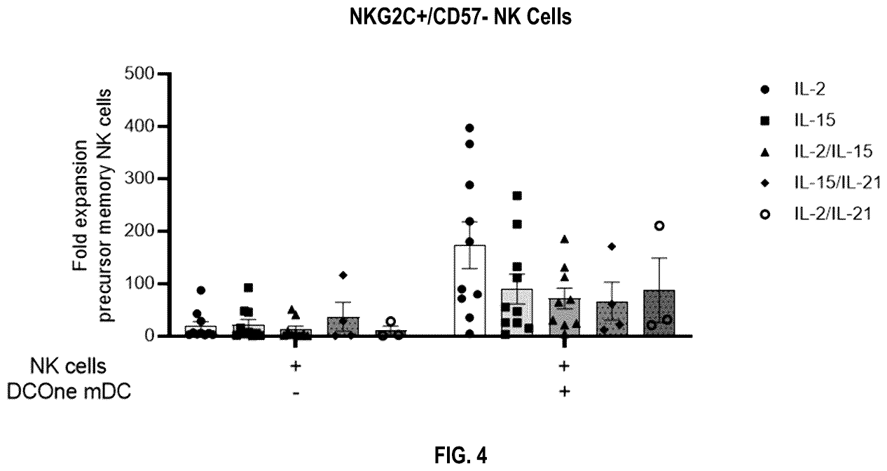

FIG. 4 shows the expansion of precursor memory (NKG2C+/CD57−) NK cells on day 14 co-cultures of NK cells and DCOne mDC, compared to the precursor memory NK cells cultured alone without DCOne mDC, in the absence and/or presence of cytokines IL-2, IL-15, or a combination of IL-2 and IL-15, IL-2 and IL-21, or IL-15 and IL-21 in a MLR assay. At day 14 cells were harvested and stained with anti-CD56, CD3, NKG2C and CD57 specific antibodies and analyzed by flow cytometry. As shown in FIG. 4, co-culturing the precursor memory NK cells with DCOne mDC significantly increases the precursor memory NK cell expansion induced by cytokines such as IL-2, IL-15, or the combinations of IL-1 and 11-15, IL-2 and IL-21, and 11-5 and IL-21.

### Example 3: DCOne mDCs Induce NK Cell Activation in Co-Culture of the NK Cells with DCOne-Derived DCs

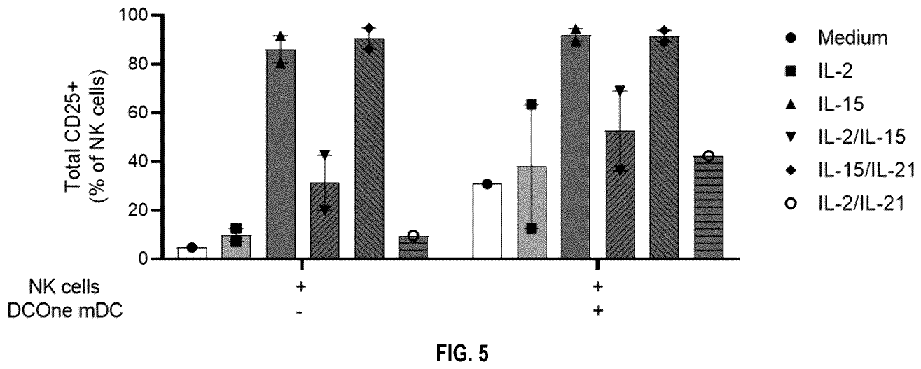

To assess NK cell activation, NK cells were co-cultured with DCOne mDCs for 7 days, compared to the NK cells cultured alone without DCOne mDC, in the absence and/or presence of cytokines IL-2, IL-15, or combination of IL-2 and IL-15, IL-2 and IL-21, or IL-15 and IL-21. At day 7 cells were harvested and stained with anti-CD56, CD3, CD25 specific antibodies and analyzed by flow cytometry (FIG. 5). As can be seen in FIG. 5, co-culturing the NK cells with DCOne mDC, significantly increases the activation of the NK cells, in the presences of IL-2, IL-15, or a combination of IL-1 and 11-15, IL-2 and IL-21, or 11-5 and IL-21.

### Example 4: DCOne mDC-NK Cell Co-Cultures Induce Increased Expansion of Memory (NKG2C+/CD57+) NK Cells are Primarily NKG2A Negative and Single KIR Positive

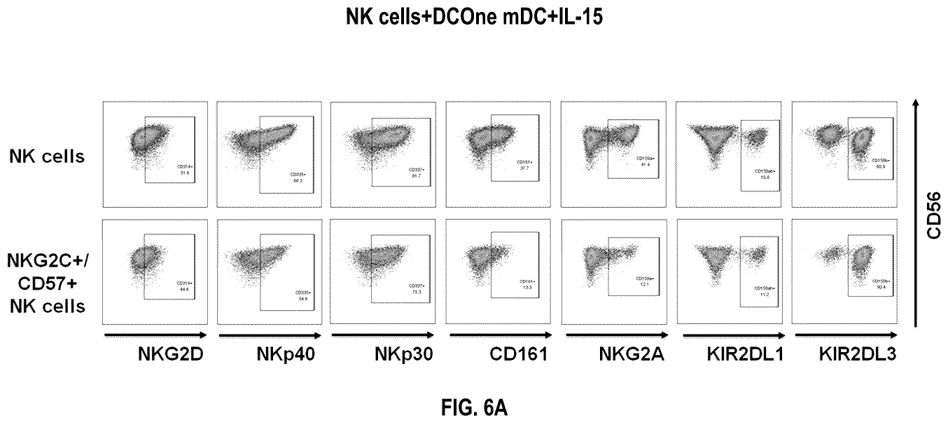

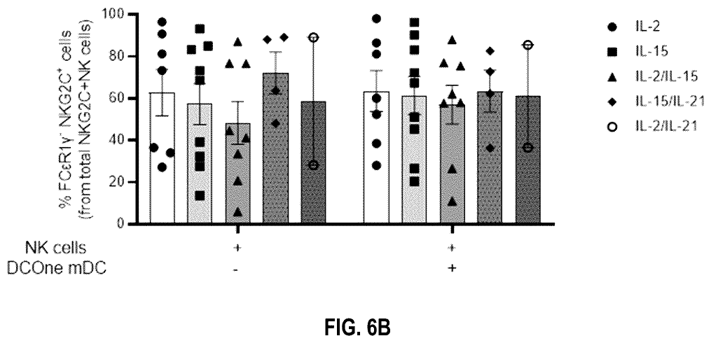

The phenotypes of expanded memory NK cells were investigated. NK cells were enriched from PBMCs isolated from buffy coats of CMV-positive healthy donors. The NK cells were co-cultured with DCOne mDC in the presence of IL-15 for 14 days. After 14 days expansion with IL-15 and DCOne-derived mDCs, flow cytometry analysis of the whole NK cell population and NKG2C+/CD57+ memory (adaptive) NK cells subpopulation. The results are show in FIG. 6A. As can be seen in FIG. 6A, the expanded adaptive NK cells were primarily NKG2A negative and single killer cell immunoglobulin-like receptor (KIR) positive.

FIG. 6B illustrates the percentage (or frequency) of FCεRIg⁻ NKG2C⁺ cells from total NKG2C⁺ cells NK cells expanded and activated 14 days using DCOne-derived mDC in the presence of cytokines IL-2, IL-15, or a combination of IL-2 and IL-15, IL-2 and IL-21, or IL-15 and IL-21. As can be seen in FIG. 6B, the expanded NKG2C⁺NK cells were primarily FCεRig negative.

### Example 5: Enhanced NK Cell Mediated Cytotoxicity Against Anti-CD38 Opsonized RAJI Tumor Cells

To assay tumor cell killing, NK cell resistant B cell lymphoblastoid RAJI cells and RAJI cells opsonized with ADCC-mediating anti-CD38 antibody were incubated with NK cells from 14-day co-cultures. After 60 minutes of incubation time, the killing of tumor cells by activated NK cells were evaluated using the GranToxiLux assay (Oncolmmunin). RAJI cells was labeled with fluorescent cell linker dye TFL4 and co-incubated with NK cells from different cocultures for 1 hour at an effector:target ratio of 10:1 in the presence of fluorogenic granzyme B substrate.

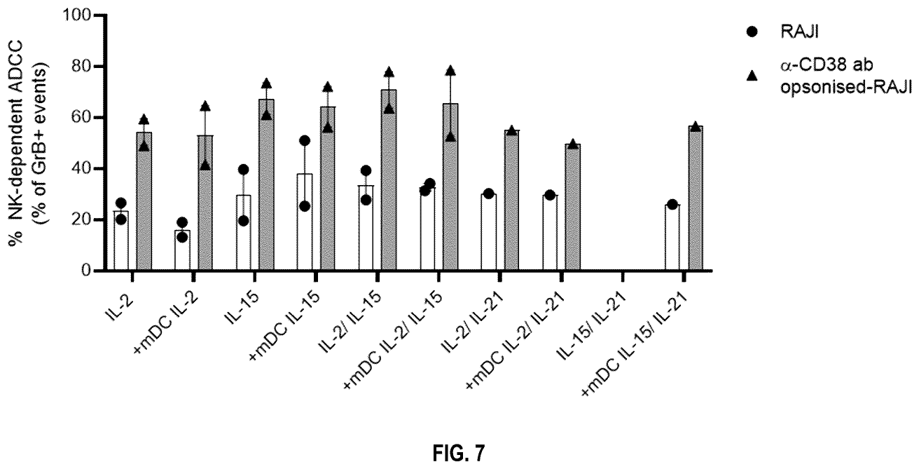

The analysis results are illustrated in FIG. 7. As shown in FIG. 7, co-incubation with activated NK cells resulted in increased detection of fluorescence in the tumor, as detected by multiparameter flow cytometry. Fluorogenic Granzyme B activity in the target tumor cells after cleavage of the granzyme B substrate was measured by using the GRANTOXILUX™ kit (Oncolmmunin, Inc., MD). This assay visualized the active amount of the cytolytic enzyme Granzyme B (GrzB) inside the tumor cells; and the binding of a fluorochrome-labelled substrate (TFL4) to active GrzB in tumor cells was visualized by flow cytometry.

### Example 6: Activating Ligands Associated with Adaptive NK Cell Expansion

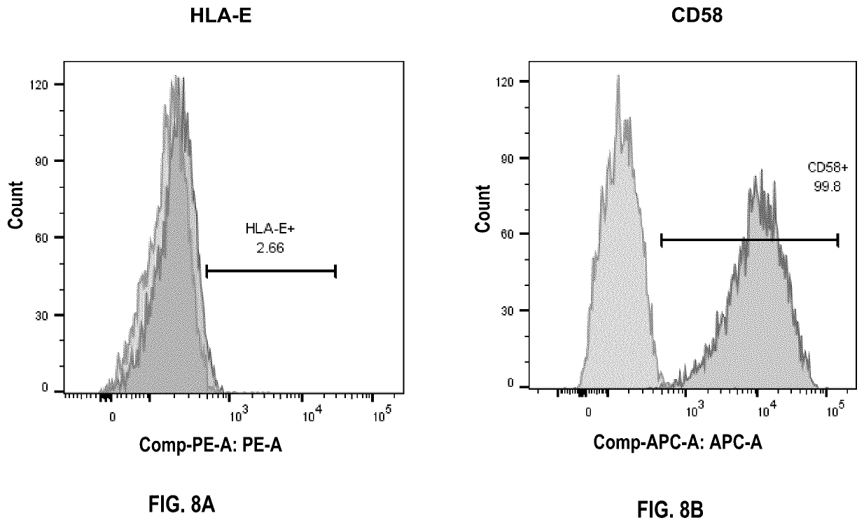

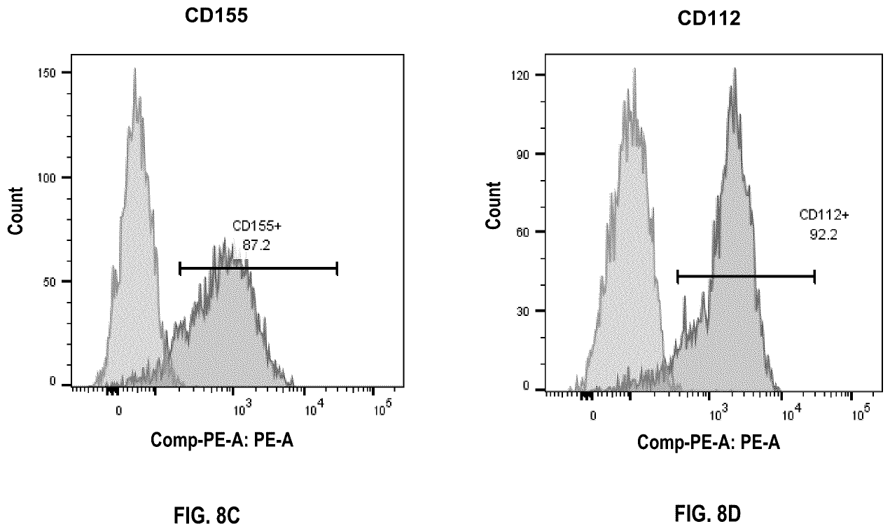

Activating ligands known to be associated with expansion of adaptive NK cells ex vivo (Liu L, et al. Cell Rep. 2016; 15(5):1088-1099; Nabekura T, et al. Immunity. 2014; 40(2):225-34) were analyzed by flow cytometry. The results are shown in FIGS. 8A-8D, which illustrate the flow cytometry analysis of activating ligands known to be associated with expansion of adaptive NK cells ex vivo. As can been seen in FIGS. 8A-8D, DCOne mDC highly express CD58, CD155, and CD112, and the activating ligands is associated with adaptive NK cell expansion.

### Example 7: NK Cells Expanded and Activated Using DCOne-Derived mDC are Highly Positive for Effector Cytokine IFN-γ after Encountering Tumor Cells

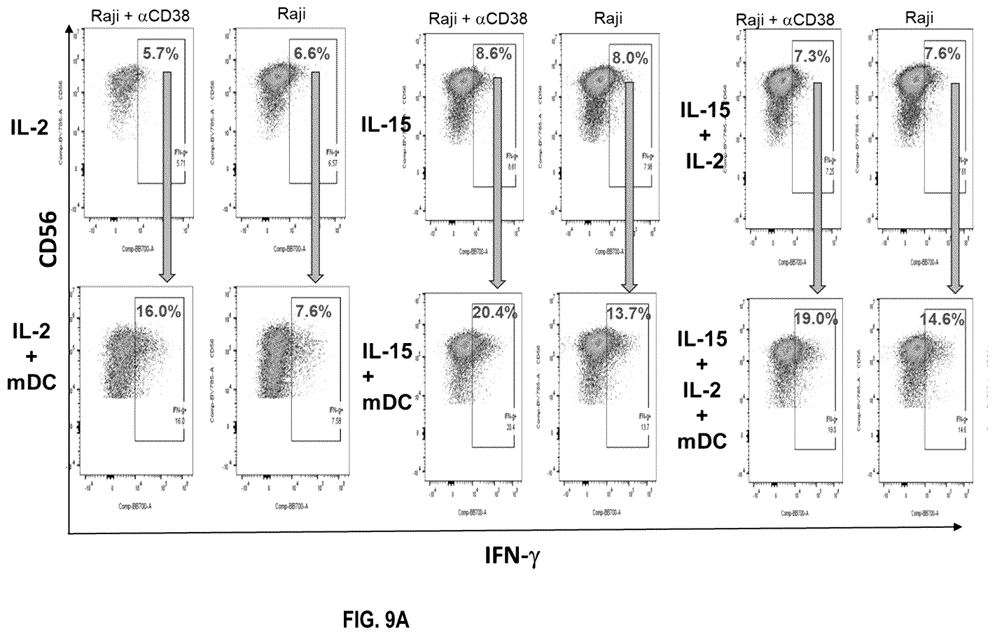

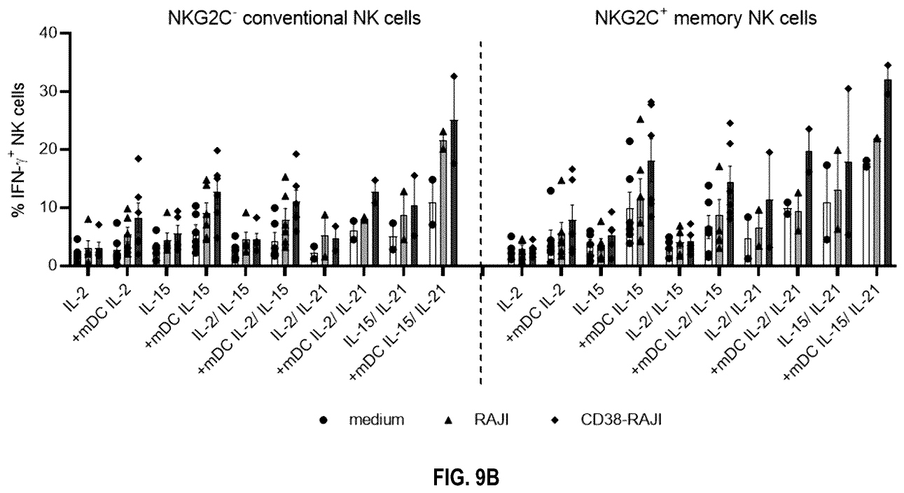

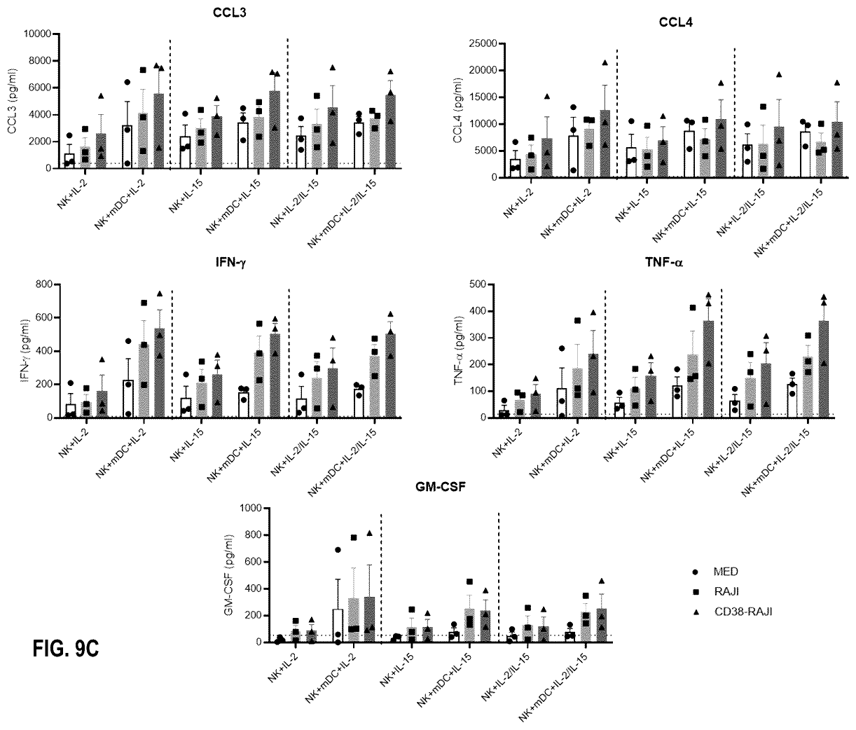

FIG. 9A-9C depicts graphs showing enhanced chemokine and cytokine production by DCOne mDC stimulated NK cells after interaction with or without anti-CD38 antibody-opsonized RAJI tumor cells. FIG. 9A is a set of exemplary flow cytometry images illustrating increased DCOne mDC stimulated (in the presence of cytokines IL-2, IL-15, or a combination of IL-2 and IL-15, IL-2 and IL-21, or IL-15 and IL-21) IFNγ positive NK cells after interaction with or without anti-CD38 opsonized RAJI tumor cells. FIG. 9B is graphic illustration of increased DCOne mDC stimulated (in the presence of cytokines IL-2, IL-15, or a combination of IL-2 and IL-15, IL-2 and IL-21, and IL-15 and IL-21) IFNγ positive NKG2C negative conventional and NKG2C positive conventional NK cells after interaction with or without anti-CD38 opsonized RAJI tumor cells. FIG. 9C depicts NK cells expanded with DCOne derived mDCs produce more CCL3, CCL4, GM-CSF, IFN-g and TNF-α upon tumor cell interaction opsonized with anti CD38 antibody.

### Example 8: NK Cells Expanded and Activated Using DCOne-Derived mDC are Superior in Persistence

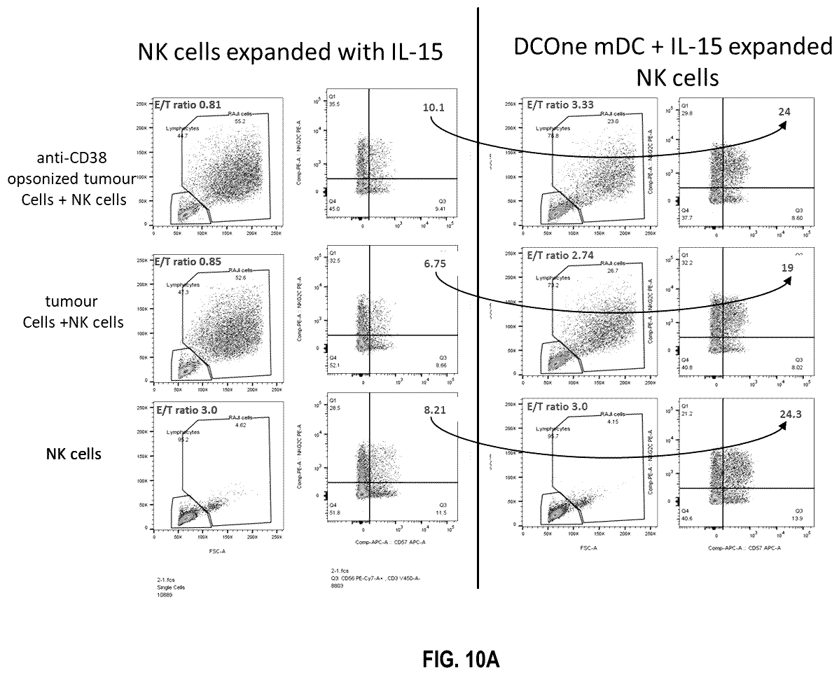

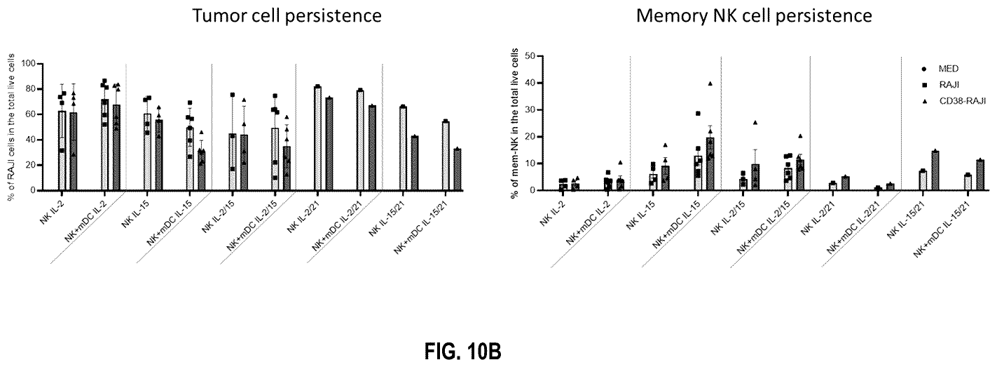

FIGS. 10A and 10B illustrates tumor cell and NK cell persistence 3-days post NK cell-tumor co-culture. NK cells expanded with DCOne mDCs in the presence of cytokines (IL-2, IL-15, or a combination of IL-2 and IL-15, IL-2 and IL-21, or IL-15 and IL-21) persist in NK-tumor cell co-cultures resulting in higher lymphocyte to tumor cell ratio.

1. A method for activating, stimulating and and/or expanding natural killer (NK) cells, comprising:
(a) contacting a population of immune cells substantially comprising natural killer (NK) cells with a population of modified cells of leukemic origin, wherein the modified cells exhibit a mature dendritic cell phenotype; and
(b) co-culturing the population of immune cells with the modified cells of leukemic origin for a period of time such that the modified cell of leukemic origin activates the natural killer (NK) cells in the population of immune cells, thereby generating an expanded population of activated natural killer (NK) cells.

2. The method of claim 1, where the co-culturing step is performed for a period of about 7 days to about 21 days, about 7 days to about 14 days, or about 14 days.

3. The method of claim 1, further comprising isolating the expanded population of activated NK cells to generate a purified population of activated NK cells.

4. The method of claim 1, wherein the NK cells:
1) express NKG2C on their cell surface;
2) are substantially comprised of memory NK cells expressing NKG2C and CD57 on their cell surface;
3) substantially comprise NKG2C+/CD57− precursors of memory NK cells; and/or
4) are low FcεRIγ expression memory NK cells, optionally FcεRIγ negative memory NK cells.

5-8. (canceled)

9. The method of claim 1, wherein:
1) least 70% of the population of immune cells is composed of NK cells; and/or
2) the population of immune cells further comprises T cells, optionally wherein the T cells constitute between 0.01% and 30% of the population of immune cell.

10. (canceled)

11. (canceled)

12. The method of claim 1, wherein:
1) the population of the immune cells is co-cultured in the presence of at least one growth factor, optionally, wherein the at least one growth factor is selected from serum, insulin, IFNγ, interleukin-2 (IL-2), IL-4, IL-7, IL-12, IL-15, IL-18, IL-21, GM-CSF, TNF-α, or any combination thereof; or
2) the population of the immune cells are co-cultured in the presence of at least one cytokine, optionally wherein the at least one cytokine is selected from IL-2, IL-15, IL-21, or combinations thereof.

13. (canceled)

14. (canceled)

15. The method of claim 12, wherein the concentration of IL-2 ranges from about 10 IU/ml to about 6000 IU/mL, the concentration of IL-15 ranges from about 5 ng/mL to about 150 ng/mL.

16. The method of claim 1, wherein the expanded population of activated NK cells is used for administering to a subject in need thereof.

17. The method of claim 1, further comprising a step of administering the expanded population of activated NK cells to a subject in need thereof.

18. The method of claim 116 or claim 17, wherein the population of immune cells are:
1) isolated from peripheral blood mononuclear cells (PBMCs) of the subject;
2) derived from induced pluripotent stem cells (iPSCs) or cord blood; or
3) are isolated from peripheral blood mononuclear cells (PBMCs) of an allogeneic third-party donor different from a subject to receive the expanded population of activated NK cells, optionally wherein the third-party donor is positive for Cytomegalovirus (CMV+).

19-21. (canceled)

22. The method of claim 1, wherein the NK cells are engineered NK cells, optionally, the engineered NK cells are NK-CAR cells or NK-TCR cells.

23. (canceled)

24. (canceled)

25. The method of claim 1, wherein the modified cell of leukemic origin comprises at least one tumor antigen selected from the group consisting of WT-1, RHAMM, PRAME, MUC-1, p53, and Survivin; and/or
wherein the modified cell of leukemic origin is CD34-positive, CD1a-positive, CD83-positive, and CD14-negative.

26. (canceled)

27. The method of claim 1 wherein the modified cell of leukemic origin comprises a co-stimulatory molecule.

28. The method of claim 27, wherein:
1) the co-stimulatory molecule is selected from an MHC class I molecule, B and T lymphocyte attenuator (BTLA), and a Toll ligand receptor;
2) the co-stimulatory molecule is selected from CD112, CD155, CD70, CD80, CD86, 4-1BBL (CD137-ligand), OX40L, CD30L, CD40, PD-L1, ICOSL, ICAM-1, lymphocyte function-associated antigen 3 (LFA3 (CD58)), K12/SECTM1, LIGHT, HLA-E, B7-H3, and CD83; or
3) the co-stimulatory molecule is selected from CD112, CD155, and/or CD58.

29. (canceled)

30. (canceled)

31. The method of claim 1 wherein:
1) the modified cell of leukemic origin further comprises a cell surface marker selected from the group consisting of DC-SIGN, Langerin, CD40, CD70, CD80, CD86, and any combination thereof;
2) the modified cell of leukemic origin is CD70-positive, CD80-positive, and CD86-positive;
3) the modified cell of leukemic origin comprises an MHC class I molecule;
4) the modified cell of leukemic origin comprises an MHC class II molecule; and/or
5) the modified cell of leukemic origin is non-proliferating.

32-35. (canceled)

36. The method of claim 1, wherein the expanded population of activated NK cells is used for administering to a subject who has been diagnosed with cancer or an infectious disease, optionally wherein the subject has previously been administered an anti-tumor IgG antibody.

37. (canceled)

38. The method of claim 1, wherein the expanded population of activated NK cells increase antibody-dependent cellular cytotoxicity (ADCC); and/or
wherein the expanded population of activated NK cells survive longer after being administered to a subject as compared to a population of NK cells that have not been in contact with the modified cell of leukemic origin prior to administration to a subject.

39. (canceled)

40. The method of claim 22, further comprising a step of introducing a chimeric antigen receptor (CAR) or an engineered T cell receptor (TCR) into the NK cells, wherein the CAR or the engineered TCR is introduced to the NK cells prior to, during, or subsequent to co-culturing the population of immune cells with the modified cells of leukemic origin;
optionally wherein the CAR or the engineered TCR is specific for one or more tumor antigens in a subject to receive the expanded population of activated NK cells.

41. (canceled)

42. The method of claim 4, wherein the activation and proliferation of the memory NK cells is effected:
1) in the absence of anti-tumor antibody-opsonized tumor cells; and/or
2) in the absence of cells expressing a ligand for NKG2C on the cell surface of the NK cells, optionally wherein the ligand for NKG2C is HLA-E.

43. (canceled)

44. (canceled)

45. The method of claim 1, wherein the expanded population of activated NK cells predominantly comprises
1) NKG2A negative and single killer Ig-like receptor (KIR) positive NK cells; and/or
2) FcεRIγ negative memory NK cells.

46. (canceled)
